SEQUENCE LISTING (67 sequences)

SEQ ID NO: 1 - DNA, 60 bp, Artificial sequence
  pIL13F1 primer
cattgctctcacttgccttggcggctttgcctccccaggccctgtgcctccctctacagc

SEQ ID NO: 2 - DNA, 60 bp, Artificial sequence
  pIL13F2 primer
gcagctagcctcatggcgcttttgttgaccacggtcattgctctcacttgccttggcggc

SEQ ID NO: 3 - DNA, 30 bp, Artificial sequence
  pIL13REcoRI primer
gagctcggatcctgaattcaaccgtccctc

SEQ ID NO: 4 - DNA, 28 bp, Artificial sequence
  pgC-F
gacacgggctaccctcactatcgagggc

SEQ ID NO: 5 - DNA, 30 bp, Artificial sequence
  pgC-R
ggtgatgttcgtcaggacctcctctaggtc

SEQ ID NO: 6 - DNA, 26 bp, Artificial sequence
  pgB1BamHI primer
gttcttcttcggtttcggatcccccg

SEQ ID NO: 7 - DNA, 26 bp, Artificial sequence
  pgB2BspEI primer
cggcatttccggaataacgcccactc

SEQ ID NO: 8 - DNA, 27 bp, Artificial sequence
  pgB3BamHI primer
cagaaaaccggatcccccaaagccgcc

SEQ ID NO: 9 - DNA, 27 bp, Artificial sequence
  pgB4BsiWI primer
gccaacacaaactcgtcgtacgggtac

SEQ ID NO: 10 - DNA, 31 bp, Artificial sequence
  first forward primer
cagttatccttaaggtctcttttgtgtggtg

SEQ ID NO: 11 - DNA, 52 bp, Artificial sequence
  first reverse primer
ccggaattccggagatcttccctcgaggaccggaaggtctttgccgcgaaag

SEQ ID NO: 12 - DNA, 43 bp, Artificial sequence
  second forward primer
ccggaattccggggtaccctggaccagctgaccgaccctccgg

SEQ ID NO: 13 - DNA, 32 bp, Artificial sequence
  second reverse primer
cggggggatgcgcagcgggagggcgtacttac

SEQ ID NO: 14 - DNA, 31 bp, Artificial sequence
  IL13 forward primer
ccgctcgagatggcgcttttgttgaccacgg

SEQ ID NO: 15 - DNA, 29 bp, Artificial sequence
  IL13 reverse primer
ggggtaccgttgaaccgtccctcgcgaaa

SEQ ID NO: 16 - DNA, 31 bp, Artificial sequence; identical to SEQ ID NO: 14
  IL 13 ORF forward primer

SEQ ID NO: 17 - DNA, 29 bp, Artificial sequence
  IL13 ORF reverse primer
ggggtaccgttgaaccgtccctcgcgaaa

SEQ ID NO: 18 - DNA, 31 bp, Artificial sequence; identical to SEQ ID NO: 14
  IL13-gD forward junction primer

SEQ ID NO: 19 - DNA, 29 bp, Artificial sequence
  IL13-gD reverse junction primer
aactgcaggttgttcggggtggccggggg

SEQ ID NO: 20 - DNA, 30 bp, Artificial sequence
  IL13 2 forward primer
aagatttgggctagcatggctttcgtttgc

SEQ ID NO: 21 - DNA, 51 bp, Artificial sequence
  IL13 2 reverse primer
tccctcgaagcttcaagcataatctggcacatcatatgtatcacagaaaaa

SEQ ID NO: 22 - DNA, 568 bp, Artificial sequence
  IL13-gC chimeric protein
gcttggtcgggaggccgcatcgaacgcacacccccatccggtggtccgtgtggaggtcgtttttcagtgcccggtctcgctttgccgggaacgctagcctcatggcgcttttgttgaccacggtcattgctctcacttgccttggcggctttgcctccccaggccctgtgcctccctctacagccctcaggtacctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaagatctgctcttacatttaaagaaactttttcgcgagggacggttgaattccacccgcatggagttccgcctccagatatggcgttactccatgggtccgtcccccccaatcgctccggc

SEQ ID NO: 23 - DNA, 350 bp, Artificial sequence
  domain of the gB open reading frame (i.e., ORF) from which the poly lysine [poly(K)] sequence was deleted
gggtcctggtggcgtcggcggctccgagttcccccggcacgcctggggtcgcggccgcgacccaggcggcgaacgggggacctgccactccggcgccgcccgcccctggccccgccccaacgggggatccgaaaccgaagaagaacagaaaaccgaaacccccaaagcgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcaga

SEQ ID NO: 24 - DNA, 655 bp, Artificial sequence
  amino-terminal sequence of IL13-gD
atggggggggctgccgccaggttgggggccgtgattttgtttgtcgtcatagtcggcctccatggggtccgcggcaaatatgccttggcggatgcctctctcaagctggccgaccccaatcgctttcgccgcaaagaccttccggtcctcgagatggcgcttttgttgaccacggtcattgctctcacttgccttggcggctttgcctccccaggccctgtgcctcccctctacagccctcagggagctcattgaggagctggtcaacatcacccagaaccagaaggctccgctctgcaatggcagcatggtttggagcatcaacctgacagctggcatgtactgtgcagccctggaatccctgatcaacgtgtcaggctgcagtgccatcgagaagacccagaggatgctgggcggattctgcccgcacaaggtctcagctgggcagttttccagcttgcatgtccgagacaccaaaatcgaggtggcccagtttgtaaaggacctgctcttacatttaaagaaactttttcgcgagggacggttcaactgaaacggtaccctggaccagctgaccgaccctccgggggtccggcgcgtgtaccacatccaggcgggcctaccggacccgttccagccccccagcctcccgatc

SEQ ID NO: 25 - DNA, 1185 bp, Artificial sequence
  gD polynucleotide
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcccggaccggctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgcccttccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggtgccgcaaatcccaccaaactggcacataccgtcgatccaggacgccgcgacgccttaccatcccccggccaccccgaacaacatgggcctgatcgccggcgcggtgggcggcagtctcctggtagccctggtcatttgcggaattgtgtactggatgcgccgccgcactcaaaaagccccaaagcgcatacgcctcccccacatccgggaagacgaccagccgtcctcgcaccagcccttgttttactag

SEQ ID NO: 26 - protein, 394 aa, Artificial sequence
  gD amino acid
MGGAAARLGAVILFVVIVGLHGVRGKYALADASLKMADPNRFRGKDLPVPDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVVPQIPPNWHIPSIQDAATPYHPPATPNNMGLIAGAVGGSLLVALVICGIVYWMRRRTQKAPKRIRLPHIREDDQPSSHQPLFY

SEQ ID NO: 27 - DNA, 2715 bp, Artificial sequence
  gB polynucleotide
atgcgccagggcgcccccgcgcgggggcgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccctggcgcccccccaacgggggacccgaaaccgaagaagaacagaaaaccgaaacccccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgccaggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgtttgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 28 - protein, 904 aa, Artificial sequence
  gB amino acid
MRQGAPARGRRWFVVWALLGLTLGVLVASAAPSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 29 - DNA, 1800 bp, Artificial sequence
  gC polynucleotide
agggcgcttggtcgggaggccgcatcgaacgcacacccccatccggtggtccgtgtggaggtcgttttcatgcccggtctcgctttgtcgggaacgctagccggtccctcgcgagggggaggcgtcgggcatggccccggggcgggtgggccttgccgtggtcctgtggagcctgttgtggctcggggcgggggtggccgggggctcggaaactgcctccaccgggcccacgatcaccgcgggagcggtgacgaacgcgagcgaggcccccacatcggggtcccccgggtcagccgccagcccggaagtcacccccacatcgaccccaaaccccaacaatgtcacacaaaacaaaaccacccccaccgagccggccagccccccaacaacccccaagcccacctccacgcccaaaagcccccccacgtccacccccgaccccaaacccaagaacaacaccacccccgccaagtcgggccgccccactaaaccccccgggcccgtgtggtgcgaccgccgcgacccattggcccggtacggctcgtgggtgcagatccgatgccggttttggaattccacccgcatggagttccgcctccagatatggcgttactccatgggtccgtcccccccaatcgctccggctcccgacctagaggaggtcctgacgaacatcaccgccccacccgggggactcctggtgtacgacagcgcccccaacctgacggacccccacgtgctctgggcggagggggccggcccgggcgccgaccctccgttgtattctgtcaccgggccgctgccgacccagcggctgattatcggcgaggtgacgcccgcgacccagggaatgtattacttggcctggggccggatggacagcccgcacgagtacgggacgtgggtgcgcgtccgcatgttccgccccccgtctctgaccctccagccccacgcggtgatggagggtcagccgttcaaggcgacgtgcacggccgccgcctactacccgcgtaaccccgtggagtttgtctggttcgaggacgaccgccaggtgtttaacccgggccagatcgacacgcagacgcacgagcaccccgacgggttcaccacagtctctaccgtgacctccgaggctgtcggcggccaggtccccccgcggaccttcacctgccagatgacgtggcaccgcgactccgtgacgttctcgcgacgcaatgccaccgggctggccctggtgctgccgcggccaaccatcaccatggaatttggggtccggcatgtggtctgcacggccggctgcgtccccgagggcgtgacgtttgcctggttcctgggggacgacccctcaccggcggctaagtcggccgttacggcccaggagtcgtgcgaccaccccgggctggctacggtccggtccaccctgcccatttcgtacgactacagcgagtacatctgtcggttgaccggatatccggccgggattcccgttctagagcaccacggcagtcaccagcccccacccagggaccccaccgagcggcaggtgatcgaggcgatcgagtgggtggggattggaatcggggttctcgcggcgggggtcctggtcgtaacggcaatcgtgtacgtcgtccgcacatcacagtcgcggcagcgtcatcggcggtaacgcgagacccccccgttacctttttaatatctatatagtttggtcccccctctatcccgcccaccgctgggcgctataaagccgccaccctctcttccctcaggtcatccttggtcgatcccgaacgacacacg

SEQ ID NO: 30 - protein, 511 aa, Artificial sequence
  gC amino acid
MAPGRVGLAVVLWGLLWLGAGVAGGSETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFDWFEDDRQVFNPGQIDTQTHEHPDGFTTVSTVTSEAVGGQVPPRTFTCQMTWHRDSVTFSRRNATGLALVLPRPTITMEFGVRHVVCTAGCVPEGVTFAWFLGDDPSPAAKSAVTAQESCDHPGLATVRSTLPISYDYSEYICRLTGYPAGIPVLEHHGSHQPPPRDPTERQVIEAIEWVGIGIGVLAAGVLVVTAIVYVVRTSQSRQRHRR

SEQ ID NO: 31 - DNA, 1282 bp, Homo sapiens
aagccacccagcctatgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctagcacttggagccaagggttcagagactcagggccccagcactaaagcagtggacaccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttccttgtatttaaatattaaatatgttagcaaagagttaatatatagaagggtaccttgaacactgggggaggggacattgaacaagttgtttcattgactatcaaactgaagccagaaataaagttggtgacagat

SEQ ID NO: 32 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 33 - DNA, 1376 bp, Artificial sequence
  IL13R 2 polynucleotide
gtaagaacactctcgtgagtctaacggtcttccggatgaaggctatttgaagtcgccataacctggtcagaagtgtgcctgtcggcggggagagaggcaatatcaaggttttaaatctcggagaaatggctttcgtttgcttggctatcggatgcttatatacctttctgataagcacaacatttggctgtacttcatcttcagacaccgagataaaagttaaccctcctcaggattttgagatagtggatcccggatacttaggttatctctatttgcaatggcaacccccactgtctctggatcattttaaggaatgcacagtggaatatgaactaaaataccgaaacattggtagtgaaacatggaagaccatcattactaagaatctacattacaaagatgggtttgatcttaacaagggcattgaagcgaagatacacacgcttttaccatggcaatgcacaaatggatcagaagttcaaagttcctgggcagaaactacttattggatatcaccacaaggaattccagaaactaaagttcaggatatggattgcgtatattacaattggcaatatttactctgttcttggaaacctggcataggtgtacttcttgataccaattacaacttgttttactggtatgagggcttggatcatgcattacagtgtgttgattacatcaaggctgatggacaaaatataggatgcagatttccctatttggaggcatcagactataaagatttctatatttgtgttaatggatcatcagagaacaagcctatcagatccagttatttcacttttcagcttcaaaatatagttaaacctttgccgccagtctatcttacttttactcgggagagttcatgtgaaattaagctgaaatggagcatacctttgggacctattccagcaaggtgttttgattatgaaattgagatcagagaagatgatactaccttggtgactgctacagttgaaaatgaaacatacaccttgaaaacaacaaatgaaacccgacaattatgctttgtagtaagaagcaaagtgaatatttattgctcagatgacggaatttggagtgagtggagtgataaacaatgctgggaaggtgaagacctatcgaagaaaactttgctacgtttctggctaccatttggtttcatcttaatattagttatatttgtaaccggtctgcttttgcgtaagccaaacacctacccaaaaatgattccagaatttttctgtgatacatgaagactttccatatcaagagacatggtattgactcaacagtttccagtcatggccaaatgttcaatatgagtctcaataaactgaatttttcttgcgaatgttgaaaaa

SEQ ID NO: 34 - protein, 380 aa, Artificial sequence
  IL13R 2 amino acid
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 35 - DNA, 2685 bp, Artificial sequence
  gB lacking polylysine tract, polynucleotide
atgcgccagggcgcccccgcgcgggggcgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccctggcgcccccccaacgggggacccgccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgccaggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgtttgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 36 - protein, 894 aa, Artificial sequence
  gB lacking polylysine tract, amino acid
MRQGAPARGRRWFVVWALLGLTLGVLVASAAPSSPGTPGVAAATQAANGGPATPAPPAPGAPPTGDPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 37 - DNA, 1626 bp, Herpes Simplex Virus-1
atggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaacgaattccacccgcatggagttccgcctccagatatggcgttactccatgggtccgtcccccccaatcgctccggctcccgacctagaggaggtcctgacgaacatcaccgccccacccgggggactcctggtgtacgacagcgcccccaacctgacggacccccacgtgctctgggcggagggggccggcccgggcgccgaccctccgttgtattctgtcaccgggccgctgccgacccagcggctgattatcggcgaggtgacgcccgcgacccagggaatgtattacttggcctggggccggatggacagcccgcacgagtacgggacgtgggtgcgcgtccgcatgttccgccccccgtctctgaccctccagccccacgcggtgatggagggtcagccgttcaaggcgacgtgcacggccgccgcctactacccgcgtaaccccgtggagtttgtctggttcgaggacgaccgccaggtgtttaacccgggccagatcgacacgcagacgcacgagcaccccgacgggttcaccacagtctctaccgtgacctccgaggctgtcggcggccaggtccccccgcggaccttcacctgccagatgacgtggcaccgcgactccgtgacgttctcgcgacgcaatgccaccgggctggccctggtgctgccgcggccaaccatcaccatggaatttggggtccggcatgtggtctgcacggccggctgcgtccccgagggcgtgacgtttgcctggttcctgggggacgacccctcaccggcggctaagtcggccgttacggcccaggagtcgtgcgaccaccccgggctggctacggtccggtccaccctgcccatttcgtacgactacagcgagtacatctgtcggttgaccggatatccggccgggattcccgttctagagcaccacggcagtcaccagcccccacccagggaccccaccgagcggcaggtgatcgaggcgatcgagtgggtggggattggaatcggggttctcgcggcgggggtcctggtcgtaacggcaatcgtgtacgtcgtccgcacatcacagtcgcggcagcgtcatcggcggtaacgcgagacccccccgttacctttttaatatctatatagtttggtcccccctctatcccgcccaccgctgggcgctataaagccgccaccctctcttccctcaggtcatccttggtcgatcccgaacgacacacg

SEQ ID NO: 38 - protein, 497 aa, Herpes Simplex Virus-1
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFNEFSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFDWFEDDRQVFNPGQIDTQTHEHPDGFTTVSTVTSEAVGGQVPPRTFTCQMTWHRDSVTFSRRNATGLALVLPRPTITMEFGVRHVVCTAGCVPEGVTFAWFLGDDPSPAAKSAVTAQESCDHPGLATVRSTLPISYDYSEYICRLTGYPAGIPVLEHHGSHQPPPRDPTERQVIEAIEWVGIGIGVLAAGVLVVTAIVYVVRTSQSRQRHRR

SEQ ID NO: 39 - DNA, 1593 bp, Artificial sequence
  gD-IL13 integration polynucleotide
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctcgagatggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaacggtaccctggaccggctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgcccttccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggtgccgcaaatcccaccaaactggcacataccgtcgatccaggacgccgcgacgccttaccatcccccggccaccccgaacaacatgggcctgatcgccggcgcggtgggcggcagtctcctggtagccctggtcatttgcggaattgtgtactggatgcgccgccgcactcaaaaagccccaaagcgcatacgcctcccccacatccgggaagacgaccagccgtcctcgcaccagcccttgttttactag

SEQ ID NO: 40 - protein, 530 aa, Artificial sequence [sequence omitted]

SEQ ID NO: 41 - DNA, 1416 bp, Artificial sequence
  IL13-gD V34S polynucleotide
atggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaacggtaccgggtcccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgcccttccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggtgccgcaaatcccaccaaactggcacataccgtcgatccaggacgccgcgacgccttaccatcccccggccaccccgaacaacatgggcctgatcgccggcgcggtgggcggcagtctcctggtagccctggtcatttgcggaattgtgtactggatgcgccgccgcactcaaaaagccccaaagcgcatacgcctcccccacatccgggaagacgaccagccgtcctcgcaccagcccttgttttactag

SEQ ID NO: 42 - protein, 471 aa, Artificial sequence
  IL13-gD V34S amino acid
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFNGTGSRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVVPQIPPNWHIPSIQDAATPYHPPATPNNMGLIAGAVGGSLLVALVICGIVYWMRRRTQKAPKRIRLPHIREDDQPSSHQPLFY

SEQ ID NO: 43 - DNA, 1416 bp, Artificial sequence
  IL13-gD V37S polynucleotide
atggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaacggtaccggggtccggcgctcgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgcccttccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggtgccgcaaatcccaccaaactggcacataccgtcgatccaggacgccgcgacgccttaccatcccccggccaccccgaacaacatgggcctgatcgccggcgcggtgggcggcagtctcctggtagccctggtcatttgcggaattgtgtactggatgcgccgccgcactcaaaaagccccaaagcgcatacgcctcccccacatccgggaagacgaccagccgtcctcgcaccagcccttgttttactag

SEQ ID NO: 44 - protein, 471 aa, Artificial sequence
  IL13-gD V37S amino acid
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFNGTGVRRSYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVVPQIPPNWHIPSIQDAATPYHPPATPNNMGLIAGAVGGSLLVALVICGIVYWMRRRTQKAPKRIRLPHIREDDQPSSHQPLFY

SEQ ID NO: 45 - DNA, 773 bp, Artificial sequence
  ATF-uPA-gD
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctcgagagcaatgaacttcatcaagttccatccaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagatggaaaaggtaccctggaccggctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaa

SEQ ID NO: 46 - DNA, 440 bp, Artificial sequence
  BD-uPA-gD
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctcgagctaaatggaggaacatgtgtgtccaacaagtacttctccaacattcactggtgcaactgcccaaagaaattcggtaccctggaccggctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaa

SEQ ID NO: 47 - DNA, 34 bp, Artificial sequence
  ATF-uPA-XhoI
ccgctcgaagcaatgaacttcatcaagttccatc

SEQ ID NO: 48 - DNA, 33 bp, Artificial sequence
  ATF-uPA-KpnI
ggggtaccttttccatctgcgcagtcatgcacc

SEQ ID NO: 49 - DNA, 29 bp, Artificial sequence
  primer for ATF-uPA-gD or BD-uPA-gD
cggaattcgatggggggggctgccgccag

SEQ ID NO: 50 - DNA, 31 bp, Artificial sequence
  primer for ATF-uPA-gD or BD-uPA-gD
aactgcagctagtaaaacaagggctggtgcg

SEQ ID NO: 51 - DNA, 2333 bp, Artificial sequence
  uPA polynucleotide
cccgggccagggtccacctgtccccgcagcgccggctcgcgccctcctgccgcagccaccgagccgccgtctagcgccccgacctcgccaccatgagagccctgctggcgcgcctgcttctctgcgtcctggtcgtgagcgactccaaaggcagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagatggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatcagcccttgctgggtgatcagcgccacacactgcttcattgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaacgacattgccttgctgaagatccgttccaaggagggcaggtgtgcgcagccatcccggactatacagaccatctgcctgccctcgatgtataacgatccccagtttggcacaagctgtgagatcactggctttggaaaagagaattctaccgactatctctatccggagcagctgaaaatgactgttgtgaagctgatttcccaccgggagtgtcagcagccccactactacggctctgaagtcaccaccaaaatgctgtgtgctgctgacccacagtggaaaacagattcctgccagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctgagggtccccagggaggaaacgggcaccacccgctttcttgctggttgtcatttttgcagtagagtcatctccatcagctgtaagaagagactgggaagataggctctgcacagatggatttgcctgtgccacccaccagggtgaacgacaatagctttaccctcaggcataggcctgggtgctggctgcccagacccctctggccaggatggaggggtggtcctgactcaacatgttactgaccagcaacttgtctttttctggactgaagcctgcaggagttaaaaagggcagggcatctcctgtgcatgggtgaagggagagccagctcccccgacggtgggcatttgtgaggcccatggttgagaaatgaataatttcccaattaggaagtgtaacagctgaggtctcttgagggagcttagccaatgtgggagcagcggtttggggagcagagacactaacgacttcagggcagggctctgatattccatgaatgtatcaggaaatatatatgtgtgtgtatgtttgcacacttgtgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtattattctgcagcatgacctgtgaccagcactgtctcagtttcactttcacatagatgtccctttcttggccagttatcccttccttttagcctagttcatccaatcctcactgggtggggtgaggaccactccttacactgaatatttatatttcactatttttatttatatttttgtaattttaaataaaagtgatcaataaaatgtgatttttctgatgaaaaaaaaaaaaaaa

SEQ ID NO: 52 - protein, 431 aa, Artificial sequence
  uPA amino acid
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 53 - DNA, 1602 bp, Artificial sequence
  ATF-uPA-gD polynucleotide
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctcgagagcaatgaacttcatcaagttccatccaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagatggaaaaggtaccctggaccggctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgcccttccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggtgccgcaaatcccaccaaactggcacataccgtcgatccaggacgccgcgacgccttaccatcccccggccaccccgaacaacatgggcctgatcgccggcgcggtgggcggcagtctcctggtagccctggtcatttgcggaattgtgtactggatgcgccgccgcactcaaaaagccccaaagcgcatacgcctcccccacatccgggaagacgaccagccgtcctcgcaccagcccttgttttactag

SEQ ID NO: 54 - protein, 534 aa, Artificial sequence
  ATF-uPA-gD amino acid
MGGAAARLGAVILFVVIVGLHGVRGKYALADASLKMADPNRFRGKDLPVLEGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKGTLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVVPQIPPNWHIPSIQDAATPYHPPATPNNMGLIAGAVGGSLLVALVICGIVYWMRRRTQKAPKRIRLPHIREDDQPSSHQPLFY

SEQ ID NO: 55 - DNA, 1269 bp, Artificial sequence
  BD-uPA-gD polynucleotide
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctcgagctaaatggaggaacatgtgtgtccaacaagtacttctccaacattcactggtgcaactgcccaaagaaattcggtaccctggaccggctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgcccttccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggtgccgcaaatcccaccaaactggcacataccgtcgatccaggacgccgcgacgccttaccatcccccggccaccccgaacaacatgggcctgatcgccggcgcggtgggcggcagtctcctggtagccctggtcatttgcggaattgtgtactggatgcgccgccgcactcaaaaagccccaaagcgcatacgcctcccccacatccgggaagacgaccagccgtcctcgcaccagcccttgttttactag

SEQ ID NO: 56 - protein, 422 aa, Artificial sequence
  BD-uPA-gD amino acid
MGGAAARLGAVILFVVIVGLHGVRGKYALADASLKMADPNRFRGKDLPVLELNGGTCVSNKYFSNIHWCNCPKKFGTLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVVPQIPPNWHIPSIQDAATPYHPPATPNNMGLIAGAVGGSLLVALVICGIVYWMRRRTQKAPKRIRLPHIREDDQPSSHQPLFY

SEQ ID NO: 57 - DNA, 25 bp, Artificial sequence
  Human uPAR reverse
ggcagtcattagcagggtgatggtg

SEQ ID NO: 58 - DNA, 25 bp, Artificial sequence
  Hamster uPAR reverse
gttgccctcgcagctgtaacactgg

SEQ ID NO: 59 - DNA, 25 bp, Artificial sequence
  uPAR-start forward
atgggtcacccgccgctgctgccgc

SEQ ID NO: 60 - DNA, 28 bp, Artificial sequence
  R5321 (pGG5414) forward primer
cggaattcatggggggggctgccgccag

SEQ ID NO: 61 - DNA, 30 bp, Artificial sequence
  R5322 (pGG5415) forward primer
cggaattcatggcgcttttgttgaccacgg

SEQ ID NO: 62 - DNA, 30 bp, Artificial sequence
  R5323 (pGG5416) forward primer
cggaattcatgagagccctgctggcgcgcc

SEQ ID NO: 63 - DNA, 33 bp, Artificial sequence
  EcoRI-PstI fragment reverse primer
aactgcagctaggcgtagtaaaccgtgatcggg

SEQ ID NO: 64 - DNA, 32 bp, Artificial sequence
  gD-A forward primer
cggaattcatgctgccccgcttcatccccgag

SEQ ID NO: 65 - DNA, 68 bp, Artificial sequence
  gD-A reverse primer
aactgcagttacaggtcctcctctgagatcagcttctgcattgatgcgttgttcggggtggccggggg

SEQ ID NO: 66 - DNA, 30 bp, Artificial sequence; identical to SEQ ID NO: 62
  uPA ATF insertion forward primer

SEQ ID NO: 67 - DNA, 33 bp, Artificial sequence
  uPA ATF insertion reverse primer
aactgcagctattttccatctgcgcagtcatgc

(19) United States
(12) Patent Application Publication
(10) Pub. No.: US 2007/0243170 A1
(43) Pub. Date: Oct. 18, 2007

(54) Targeting of Herpes Simplex Virus to Specific Receptors

(75) Inventors: Bernard Roizman, Chicago, IL (US); Guoying Zhou, Chicago, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE UNIVERSITY OF CHICAGO, Chicago, IL (US)

(21) Appl. No.: 11/677,026

(22) Filed: Feb. 20, 2007

Related U.S. Application Data
(63) Continuation-in-part of application No. PCT/US06/32291, filed on Aug. 18, 2006.
(63) Continuation-in-part of application No. 11/215,636, filed on Aug. 30, 2005.
Application No. 11/215,636 is a continuation-in-part of application No. 10/530,774, filed on Nov. 17, 2005.
(60) Provisional application No. 60/709,597, filed on Aug. 19, 2005.
(60) Provisional application No. 60/416,716, filed on Oct. 7, 2002.

(51) Int. Cl. A61K 35/76; C12N 7/00

(52) U.S. Cl. 424/93.6; 435/235.1

(57) ABSTRACT

The invention relates to engineered herpes simplex virus (HSV) particles that are targeted to one or more specific binding pair members, such as receptors. Also, recombinant vectors for producing such HSV particles are provided. By reducing the affinity of HSV for its natural receptor(s) and increasing the affinity for a selected receptor, the HSV particles of the invention are useful for targeting cells that express the selected receptor, which itself may be a product of genetic engineering. The ability to selectively target cells renders the HSV particles particularly useful in selectively diagnosing, treating, and imaging cells bearing the selected binding pair member, such as a receptor. The invention also provides for polynucleotide-based therapy to cells bearing the selected binding pair member such as a receptor.

This application is a continuation-in-part application of U.S. Ser. No. 11/215,636, filed Aug. 30, 2005, which is a continuation-in-part of U.S. Ser. No. 10/530,774, filed Apr. 7, 2005, which is the US national phase of PCT/US03/31598, filed Oct. 6, 2003, which claims the priority benefit of U.S. Ser. No. 60/416,716, filed Oct. 7, 2002. This application also claims priority to PCT/US2006/032291, filed Aug. 18, 2006, which claims the priority benefit of U.S. Ser. No. 60/709,597, filed Aug. 19, 2005.

### GOVERNMENT INTERESTS

This invention was made with U.S. government support under CA 115662, CA 83939, CA 71933, CA 78766 and CA 88860 awarded by the National Institutes of Health. The U.S. government has certain rights in the invention

### BACKGROUND OF THE INVENTION

A steady rate of healthcare advances has led to continuing improvement in the health and quality of life for humans and animals. Nevertheless, a variety of diseases, disorders, and conditions have largely eluded the best efforts at prevention or treatment. Chief among these maladies is the loss of cell-cycle control that frequently results in the undesirable cell proliferation characteristic of cancer in its many forms, such as malignant glioma. Malignant gliomas are devastating brain tumors that afflict animals such as humans. The average life span after diagnosis is less than one year and few patients have been reported to survive five years. Furthermore, none of the conventional anti-cancer therapies has been successful in significantly prolonging the lifespan of patients with this disease. In recent years there have been numerous attempts to use genetically engineered herpes simplex viruses (HSV) as oncolytic agents to treat malignant gliomas. Because wild-type viruses are highly virulent, the viruses used in preclinical evaluations and in phase-1 clinical studies have been thoroughly attenuated. While several deletion mutants have been tested, the mutants that reached clinical trials lacked the γ₁34.5 gene encoding infected cell protein 34.5 (ICP34.5) and optionally, the U_{L}39 gene encoding the large subunit of ribonucleotide reductase.

These attenuated HSV viruses, however, have been imperfectly engineered as oncolytic agents. One advantage of these mutant viruses is that they have a significantly reduced capacity to replicate in normal, non-dividing cells in vivo. Viral ribonucleotide reductase is an essential gene for viral replication in resting cells and, hence, the U_{L}39 mutant virus is dysfunctional in the normal environment of the central nervous system (Simard et al. 1995). The major function of ICP34.5 is to preclude the shutoff of protein synthesis caused by activation of protein kinase R in infected cells. Once activated, this enzyme phosphorylates the α subunit of translation initiation factor 2 (eIF2α), resulting in complete cessation of translation. Mutants lacking the γ₁34.5 genes are highly attenuated because the lytic life cycle is completely blocked in an interferon⁺ cellular background. In contrast, γ₁34.5 mutants are nearly as virulent as wild-type virus in mice lacking interferon receptor. Although mutants deleted in both γ₁34.5 and U_{L}39 are not significantly more attenuated than those lacking the γ₁34.5 genes, such mutants do provide added insurance in the form of a reduced risk of reversion.

A significant disadvantage of these mutant HSV viruses is their poor replication, even in dividing cells. In experimental animal systems, the mutant viruses do not exhibit sustained lytic life cycles, with the loss of a potentially amplified response to a given therapeutic dose of the virus that would be expected upon re-infection of tumor cells by the multiplied viral progeny. Consequently, maximum killing of tumors cells requires high doses of virus. Given the poor growth of these mutant HSV viruses, even in dividing cells, production of virus pools large enough to yield efficacious inocula of >10⁹ plaque forming units (PFU) has remained a major obstacle. Moreover, indiscriminate binding of virus to non-tumor cells further diminishes the effectiveness of HSV virus dosages because mis-targeted viral particles do not contribute to the desired beneficial therapeutic effect of tumor cell destruction. One approach to overcoming these obstacles is to achieve a more thorough understanding of the HSV lytic life cycle and thereby facilitate the development of HSV mutants tailored for use as targeted therapeutic agents, such as targeted oncolytic agents.

HSV enters host cells using a two-step mechanism. The first step of entry is HSV attachment to the cell surface. Common receptors for viral entry are nectin-1, HveA, and a specific O-linked sulfated proteoglycan. This step is initiated by glycoproteins B and C (gB and gC), which project from the viral envelope, attaching to heparan sulfate proteoglycans on host cell surfaces. The gB and gC domains interacting with heparan sulfate have been mapped at the sequence level (Laquerre et al. 1998). Following this initial attachment, viral glycoprotein D (gD, 369 amino acids) interacts with one of several receptors. Of these gD receptors, two are particularly important for entry (Spear et al, 2000). One receptor, designated HveA (formerly, HveM), is a member of the family of receptors for tumor necrosis proteins. A second receptor, designated Nectin-1 (HveC), is a member of the nectin family of proteins, structurally related to the immunoglobulin superfamily, which serve as intercellular connectors (Campadelli-Fiume et al. 2000). The second step of HSV entry into a cell is fusion of the viral envelope with the plasma membrane of the cell. To effect fusion, gD, when bound to its receptor, recruits glycoproteins B, H and L, which results in fusion of the envelope with the plasma membrane.

Additional understanding of HSV infection has come from recent studies that have lent significance to an old observation that gD interacts with the cation-independent mannose 6 phosphate receptor, contributing to the accumulation of HSV in endosomes. Endocytosis of viral particles results in particle degradation by lysosomal enzymes, but the cells succumb as a consequence of the degradation of cellular DNA by lysosomal DNase. HSV gD blocks this apoptotic pathway to cell death through its interaction with the mannose 6 phosphate receptor. In fact, transduction of cells with specific domains of gD, treatment with chloroquine or overexpression of cation-independent mannose 6-phosphate receptor prevents cell death (15-17). The domains required for blocking cell death have been mapped by insertional mutagenesis to several sites of gD (17). Thus, gD interacts with HveA, nectins, the mannose 6 phosphate receptor, and at least one of the complex of viral glycoproteins involved in the fusion of HSV with the plasma membrane.

In an attempt to target HSV-1 infection to specific cells, a recombinant HSV having a chimeric protein comprising gC and erythropoietin (EPO) on its surface was constructed. Although the recombinant virus bound to cells expressing EPO receptor and endocytosis of the virus occurred, successful infection of these EPO-receptor expressing cells did not occur.

Accordingly, a need continues to exist in the art for viral therapeutic agents exhibiting improved targeting capacities while retaining sufficient capacity to infect to be therapeutically useful. Ideally, suitable viruses would be therapeutic agents, such as oncolytic agents, themselves as well as providing a targeting vehicle or vector for the controlled delivery of polynucleotide coding regions useful as therapeutic agents. Another need in the art is for targeted agents useful in diagnostic applications as, e.g., imaging agents or targeted vehicles for imaging agents.

### SUMMARY

The invention satisfies the aforementioned need in the art by providing viral forms suitable for use as therapeutic and diagnostic agents themselves, as well as providing a ready vehicle for the delivery of therapeutic or diagnostic polynucleotide coding regions to cells. These viral forms are modified viruses of the Herpesviridae family of viruses, and are preferably derived from herpes simplex virus type 1 or type 2. The invention provides a method of making virus particles with a novel ligand (or binding pair member), and making said particles totally dependent on a receptor of the ligand (or binding pair member) for entry into targeted cells.

Disclosed herein are methods to modify the surface of, e.g., an HSV virus particle in a manner that targets the virus to a specific receptor present on the surface of a cell of choice, typically a cell in need of therapy or a cell whose presence provides information of diagnostic value. The invention provides viral particles, e.g., HSV particles, having a reduced affinity for their natural cell-surface receptor(s), and methods for producing and using such particles, which minimizes or eliminates the problem of reduced efficiency associated with the mis-targeting of therapeutic and diagnostic viruses. Additionally, the invention provides viral particles, e.g., HSV particles, that exhibit specific affinity for a cell surface component that is not a natural viral receptor and that is present solely or predominantly on a given target cell, as well as methods for producing and using such viruses. Modified viral particles (e.g., HSV) having increased affinity for a cell surface component associated with one or more target cells exhibit improved targeting capabilities relative to known viral particles. The modified HSV particles have reduced indiscriminate binding, thereby minimizing sequestration of viral dosages away from the target cells. The invention further provides modified viral particles, such as modified HSV particles, that have both a reduced affinity for natural viral receptors and an increased affinity for a cell surface component associated with a particular target cell(s), with the modified viral particle effectively recruiting that cell surface component for use as a viral receptor. Other benefits of the modified viruses are described herein and will be apparent to those of skill in the art upon review of this disclosure.

In one aspect, the invention provides a herpes simplex virus comprising a first polynucleotide encoding a gD polypeptide fragment comprising about amino acids 244-394 of SEQ ID NO:26 (preferably amino acids 244-394 of SEQ ID NO:26, corresponding to amino acids 219-369 of mature gD) and a second polynucleotide encoding a targeting peptide, wherein said targeting peptide specifically interacts with the gD polypeptide fragment in addition to specifically interacting with its binding partner, the target. The viruses according to the invention provide for a simplified modular approach to the construction of re-targeted HSV, requiring a coding region for any peptide capable of specifically interacting with a target of interest. Typically, the targeting peptide is a member of a binding pair or is an antibody product specifically recognizing a target of interest, which is typically a peptide target. In some embodiments, the targeting peptide is a urokinase plasminogen activator peptide that specifically interacts with urokinase plasminogen activator receptor.

A related aspect of the invention is drawn to the herpes simplex virus described above, wherein the second polynucleotide further encodes a second gD polypeptide fragment comprising at least about 28 (preferably 28) contiguous amino acids of SEQ ID NO:26 and having a C-terminus at about position 60 (preferably at position 60) of SEQ ID NO:26. In an embodiment, the second gD polypeptide fragment comprises, or consists essentially of, amino acids 33-60 of SEQ ID NO:26. In some embodiments, the herpes simplex virus comprises a second polynucleotide wherein the second polynucleotide further encodes a peptide linker interposed between said targeting peptide and said second gD polypeptide fragment. An exemplary peptide linker is (Gly₄Ser)ₙ, wherein n is an integer between 3 and 5, inclusive.

Another aspect of the invention is drawn to a herpes simplex virus comprising a polynucleotide encoding a gD polypeptide fragment comprising about amino acids 244-394 (preferably amino acids 244-394) of SEQ ID NO:26 (corresponding to residues 219-369 of mature gD) and a targeting peptide, wherein the targeting peptide is a member of a binding pair that specifically interacts with the other member of the binding pair. In some embodiments, the polynucleotide further encodes a peptide linker interposed between the targeting peptide and the gD polypeptide fragment. Again, an exemplary peptide linker is (Gly₄Ser)ₙ, wherein n is an integer between 3 and 5, inclusive. In some embodiments according to this aspect, the polynucleotide further encodes a second gD polypeptide fragment comprising at least about 28 (preferably 28) contiguous amino acids of SEQ ID NO:26 and having a C-terminus at about position 60 (preferably position 60) of SEQ ID NO:26. In some embodiments, the second gD polypeptide fragment comprises, or consists essentially of, amino acids 33-60 of SEQ ID NO:26. Consistent with other aspects of the invention, the polynucleotide may further encode a peptide linker interposed between the gD polypeptide fragment and the second gD polypeptide fragment, and/or the same or a different peptide linker may be interposed between the targeting peptide and the gD polypeptide fragment. In these various product aspects of the invention, exemplary peptide linkers are linkers conforming to the pattern of (Gly₄Ser)ₙ, wherein n is an integer between 3 and 5, inclusive.

As noted above, the products according to the invention provide a simplified modular approach to re-targeted HSV in which a targeting peptide is encoded by a polynucleotide. Increasing the versatility of the invention are constructs that further provide an engineered pair of interacting domains/motifs to ensure that the N-terminally disposed interaction domain of gD (e.g., gD residues 33-60) interacts with the C-terminally disposed fusogenic domain of gD (e.g., gD residues 219-369, or 244-394 of SEQ ID NO:26). A wide variety of interacting domains/motifs are known in the art or can be generated (e.g., antibodies elicited to a particular peptide). One member of a pair of interacting domains/motifs is fused to the fusion peptide comprising a targeting peptide and the N-terminally disposed interaction domain of gD on a single polypeptide or polypeptide fragment (e.g., targeting peptide—interacting domain—gD interaction domain); the other member of the pair of interacting domains/motifs is translationally fused to the fusogenic domain of gD on a polypeptide or polypeptide fragment (e.g., interacting domain partner—gD fusogenic domain). The two fusion polypeptides or polypeptide fragments, moreover, can be present on a single protein chain or on distinct protein chains. Given the freedom from constraints placed upon the targeting peptide by the design of the invention, it is apparent that any polynucleotide encoding any targeting peptide would be suitable for use in the invention. In some embodiments, the targeting peptide is selected from the group consisting of a urokinase plasminogen activator peptide fragment and an interleukin 13 peptide fragment, wherein said peptide fragment specifically interacts with its binding partner. In other embodiments, the targeting peptide comprises an antibody variable domain, wherein the peptide specifically interacts with its binding partner.

Another aspect of the invention is a pharmaceutical composition comprising the herpes simplex virus described herein, and a pharmaceutically acceptable excipient, carrier or diluent. A related aspect of the invention is drawn to a kit comprising the herpes simplex virus described herein and a label providing instruction for administration of the virus.

Yet another aspect of the invention is a method of producing a herpes simplex virus as described herein comprising (a) contacting a permissive host cell with a herpes simplex virus as described herein; (b) incubating the host cell; and (c) recovering the herpes simplex virus.

Another aspect of the invention is a method of treating a condition in an organism characterized by the presence of a deleterious cell in the organism comprising administering a therapeutically effective amount of a herpes simplex virus as described herein to the organism. Conditions contemplated as amenable to treatment include cancer, autoimmune disease and any other hyperproliferative cell disorder. In some embodiments, the condition is cancer.

Still another aspect of the invention is a method of screening for a targeting peptide comprising (a) preparing a plurality of herpes simplex viruses as described herein wherein the plurality of viruses comprises a plurality of targeting peptide coding regions; (b) contacting the targeting peptides with a target peptide; and (c) measuring the interaction of the targeting peptides and the target peptide, thereby identifying a targeting peptide binding partner of the target peptide. In some embodiments, the target peptide is associated with a cell. In such embodiments, measurements of peptide interactions include measurements of HSV entry into the cell using any technique known in the art, such as viral titering, uptake of labeled virus and the like.

In another aspect, the invention provides a recombinant herpes simplex virus (HSV) particle having at least one protein on its surface, comprising: (a) an altered gD, wherein the alteration reduces binding of gD to one or more of its cellular receptors, said alteration comprising (i) a heterologous peptide ligand on the surface of the recombinant HSV particle forming a fusion protein with the altered gD; and (ii) an amino acid alteration; wherein said recombinant HSV particle preferentially binds to cells expressing a binding partner to said heterologous peptide ligand. In some embodiments, these particles preferentially bind to target cells (cell expressing a binding partner) in whole or part due to the greater frequency of the binding partner on the surface of the cell relative to any natural HSV binding proteins on the surface of that cell. In some embodiments, the recombinant HSV particle further comprises an altered viral surface protein, wherein the alteration reduces binding of the viral surface protein to a sulfated proteoglycan. Such recombinant herpes simplex virus (HSV) particles comprise a virus surface protein altered to reduce the wild-type level of binding of that protein to a sulfated proteoglycan on the surface of a cell and an altered gD. The altered gD exhibits a reduced binding to one or more of the natural cellular receptors for gD; the altered gD is also fused to a heterologous peptide ligand (or binding pair member) having a binding partner, e.g., a peptide ligand receptor, found on the surface of a cell. Stated in the alternative, this aspect of the invention provides a recombinant herpes simplex virus (HSV) particle having at least one protein on its surface, comprising: (a) an altered viral surface protein, wherein the alteration reduces binding of the viral surface protein to a sulfated proteoglycan; and (b) an altered gD, wherein the alteration reduces binding of gD to one or more of its cellular receptors, the alteration comprising (i) a heterologous peptide ligand (or binding pair member) on the surface of the recombinant HSV particle forming a fusion protein with the altered gD; and (ii) an amino acid alteration; wherein the recombinant HSV particle preferentially binds to cells expressing a binding partner to the heterologous peptide ligand (or binding pair member).

The invention comprehends a recombinant HSV particle wherein the amino acid alteration is selected from the group consisting of an amino acid deletion, an amino acid substitution and an amino acid insertion. A preferred site for the amino acid alteration is amino acid position 34 of gD. Exemplary recombinant HSV particles according to the invention include HSV R5141 and HSV R5161, each described below.

Contemplated amino acid alterations include insertions or deletions of 1-10 amino acids, such as insertions or deletions of 1-5 amino acids. Exemplary insertions occur immediately upstream (N-terminal) or downstream (C-terminal) to amino acid position 34 of gD. Exemplary deletions include amino acid position 34 of gD. For alterations comprising amino acid substitutions, 1-10 amino acids are substituted, such as substitutions of 1-5 amino acids. Non-contiguous (dispersed) or contiguous amino acid substitutions are contemplated. In some embodiments, conservative amino acids known in the art are substituted. Exemplary amino acid substitutions include single amino acid substitutions for the valine at position 34 of mature gD (position 59 of holo-gD, see SEQ ID NO:26). Preferably, substitutions for Val34 will be V34S or a conservative substitution for the native Val at position 34 of mature gD.

The altered gD, moreover, reduces binding of the recombinant HSV particle to at least one HSV entry mediator (Hve) cell-surface protein, such as an Hve selected from the group consisting of HveA (formerly, HveM) and Nectin-1 (HveC). Further, the recombinant HSV particles of the invention include particles wherein the altered viral surface protein is selected from the group consisting of gB and gC. In some embodiments, the altered viral surface protein, preferably selected from the group of gB and gC, forms a fusion protein with a heterologous peptide ligand. In some embodiments, the binding partner is a cell surface receptor for the heterologous peptide ligand.

The preferential binding of the recombinant HSV particles of the invention results in a detectable variation in effective binding of the particle to the cells being compared. By “effective binding” is meant either sufficiently stable binding to permit detection of binding or binding sufficient to result in productive infection of the cell. In preferred embodiments, the preferential binding is such that the recombinant HSV particles bind only to one of the cell types being compared (e.g., cancer cells compared to healthy cells of the same type). Suitable cells include any hyperproliferative cell type, such as a cancer cell. A cancer cell, in turn, includes a tumor cell, e.g., a malignant gliomal cell.

The invention comprehends recombinant HSV particles wherein the heterologous peptide ligand (or binding pair member) is any ligand (or binding pair member) for which a cell surface binding partner exists. Preferably, heterologous peptide ligands have specific cell surface binding partners, e.g., ligand receptors, that are preferentially exhibited on the surface of a target cell. More preferably, the cell surface binding partner is only exhibited on the surface of a target cell, when compared to the cells in an organism containing the target cell. Exemplary heterologous peptide ligands that include cytokines, such as IL113, and fragments, variants and derivatives thereof, provided that the ligand retains the capacity of binding to a cell-surface binding partner. An exemplary binding pair member contemplated as suitable for each aspect of the invention is a single-chain antibody, for which a binding partner would include an antigen thereof, or a fragment, derivative or variant thereof that retains the capacity to bind to the single-chain antibody.

Another aspect of the invention is drawn to the recombinant HSV particle described above, wherein a polynucleotide encoding the fusion protein is joined to a heterologous coding region for a leader sequence. In this context, “heterologous” means that the leader sequence is not found naturally associated with the upstream or 5′ coding region participating in the fusion. Exemplary leader sequences include HSV leader sequences, e.g., an HSV gD leader sequence. In a related aspect, the invention provides the recombinant HSV particle described above, wherein a polynucleotide encoding the fusion protein is joined to a heterologous expression control element, such as a heterologous promoter (a promoter not naturally found in association with the polynucleotide coding region fused upstream or 5′ in the fusion), a heterologous enhancer, or expression factor binding site known in the art.

Another aspect of the invention provides a pharmaceutical composition comprising a recombinant HSV particle described above and a pharmaceutically acceptable carrier, diluent, or excipient. Any pharmaceutical carrier, diluent or excipient known in the art is contemplated. A related aspect of the invention provides a kit comprising the pharmaceutical composition and a set of instructions for administering the composition to a subject in need. In each of these aspects of the invention, i.e., the pharmaceutical compositions and the kits, the heterologous peptide ligands (or binding pair members) and cell-surface binding partners described in the context of describing the recombinant HSV particles are contemplated.

Yet another aspect of the invention provides a method of targeting a recombinant HSV particle to a cell comprising (a) identifying a binding pair member, such as a ligand for a ligand binding partner, exhibited on the surface of a target cell; and (b) creating an HSV particle as described herein, wherein the ligand or, more generally, the binding pair member, binds to the binding partner exhibited on the surface of the target cell. In some preferred embodiments of this aspect of the invention, the altered viral surface protein is selected from the group consisting of gB and gC. In some embodiments, the alteration to gD reduces binding of gD to at least one cellular receptor for gD selected from the group consisting of HveA and Nectin-1. In preferred embodiments, the altered gD has a conservative substitution at position 34 of gD, such as a V34S substitution. A second fusion protein, joining the ligand (or binding pair member) and either of gB or gC, is also contemplated in some embodiments. Suitable cells for targeting include any hyperproliferative cell, such as a cancer cell, including tumor cells (e.g., malignant gliomal cells). Any of the heterologous peptide ligands (or binding pair members) and cell-surface binding partners described above in the context of describing the recombinant HSV particles is suitable for use in the method.

Another aspect of the invention is drawn to a method of imaging a cell comprising: (a) contacting the cell with a recombinant HSV particle as described above, the recombinant HSV particle further comprising a coding region for a marker protein; and (b) detecting the presence of the marker protein. Any type of cell exhibiting a cell-surface binding partner for a ligand (or binding pair member) fusible to HSV gD is suitable for use in this aspect of the invention, such as a cancer cell. Using cancer cells as an example, the method is useful provided that the binding partner is present at a higher number on the cancer cell as compared to a non-cancerous cell of the same type. Any known marker protein is useful in this aspect of the invention, e.g., a marker protein selected from the group consisting of thymidine kinase, green fluorescent protein, and luciferase. In preferred embodiments, the altered gD exhibits an amino acid substitution of V34S. Any of the heterologous peptide ligands (or binding pair members) and cell-surface binding partners described above in the context of describing the recombinant HSV particles is suitable for use in the method.

Another aspect of the invention provides a method of treating a cell-based disease comprising delivering a therapeutically effective amount of a recombinant HSV particle as described herein to a subject in need. A related aspect is the use of a recombinant HSV particle as described above in the preparation of a medicament for the treatment of a cell-based disease. A therapeutically effective amount of a recombinant HSV particle is that amount that produces the desired therapeutic effect, as would be understood and readily determinable by those of skill in the art. Any cell-based disease known or reasonably suspected to be amenable to treatment with a specifically targeted HSV is contemplated, e.g., a cell hyperproliferation disease such as cancer. Any of the heterologous peptide ligands (or binding pair members) and cell-surface binding partners described above in the context of describing the recombinant HSV particles is suitable for use in the method.

In a related aspect, the invention provides a method of ameliorating a symptom associated with a disease comprising administering a therapeutically effective amount of a recombinant HSV particle described above to a subject in need. Another aspect is drawn to the use of a recombinant HSV particle as described above in the preparation of a medicament for ameliorating a symptom associated with a disease in a subject in need. Again, any disease known or reasonably suspected to have a symptom amenable to application of a specifically targeted HSV is contemplated, including any disease characterized by hyperproliferative cells, such as cancer. Any of the heterologous peptide ligands (or binding pair members) and cell-surface binding partners described above in the context of describing the recombinant HSV particles is suitable for use in the method.

Another aspect of the invention is directed to a method of delivering a therapeutically useful peptide to a cell comprising: (a) inserting a therapeutically useful polynucleotide, such as an expression control element, an rDNA, or a coding region for a therapeutically useful peptide, into the DNA of a recombinant HSV particle as described above, thereby producing a recombinant HSV clone; and (b) delivering a therapeutically effective amount of the recombinant HSV clone to the cell. In a related aspect, the invention provides for the use of a recombinant HSV clone comprising a recombinant HSV particle according to claim 1 in the preparation of a medicament for delivering a therapeutically useful peptide to a cell comprising inserting a coding region for a therapeutically useful peptide into the DNA of the recombinant HSV particle, thereby producing the recombinant HSV clone. Each of the method and use comprehends delivery of the recombinant HSV clone in vivo, ex vivo, or in vitro. Any of the heterologous peptide ligands (or binding pair members) and cell-surface binding partners described above in the context of describing the recombinant HSV particles is suitable for use in the method or use.

Another aspect of the invention provides a method of killing a target cell, comprising contacting the target cell with a recombinant HSV particle as described above. A related aspect is the use of a recombinant HSV particle as described above in the preparation of a medicament for killing a target cell by contacting the target cell with the recombinant HSV particle. In preferred embodiments of either the method or the use, the recombinant HSV particle has an altered gD in which the V34S substitution is found. Any of the heterologous peptide ligands (or binding pair members) and cell-surface binding partners described above in the context of describing the recombinant HSV particles is suitable for use in the method or use.

In each of the above-described aspects of the invention, it is preferred that gD, or a portion thereof, maintains its membrane fusion properties, but has reduced capacity to bind HveA and/or Nectin-1.

Other features and advantages of the invention will be better understood by reference to the brief description of the drawing and the description of the illustrative embodiments that follow.

### BRIEF DESCRIPTION OF THE DRAWING

FIG. 1. Schematic representation of the HSV-1 (F) genome and gene manipulations in glycoprotein C (gC) (FIG. 1A), glycoprotein B (gB) (FIG. 1B), and glycoprotein D (gD) (FIG. 1C). Line 1, sequence arrangement of the HSV-1 genome. The rectangular boxes represent the inverted repeat sequences ab and b′a′ flanking the unique long (U_{L}) sequence, and inverted repeat c‘a’ and ca flanking the unique short (U_{S}) sequence. Line 2, sequence arrangement of domains of the glycoprotein C; the signal peptide (SP) domain and heparan sulfate (HS)-binding domain of gC are highlighted. Line 3, human IL13 with signal peptide that replaced the N-terminal segment of 148 amino acids of gC. Line 4, sequence arrangement of the poly-lysine domain of gB. Line 5, schematic representation of a recombinant HSV-1(F) genome, in which the N-terminal domain of gC was replaced with IL13 and the polylysine domain (from codon 68 to codon 77) of gB was deleted. Line 6, sequence arrangement of glycoprotein J (gJ), glycoprotein D (gD), and glycoprotein I (gI) in U_{S}. Line 7, replacement of gD with the immediate early promoter of CMV in order to enable the expression of gI. Line 8, schematic representation of recombinant HSV-1 (F) genome, in which the N-terminal domain of gC was replaced with IL13, the poly-lysine domain of gB was deleted, and IL13 was inserted after amino acid 24 of gD (FIG. 1D). Line 9, a polylinker XhoI-BglII-EcoRI-KpnI was inserted after amino acid 24 of gD, with IL13 inserted into the XhoI and KpnI sites of gD.

FIG. 2. Amino acid sequence alignment of IL13-gC, IL13-gD junction sequence, and the HS binding domain of gB. FIG. 2A. The amino-terminal sequence of IL13-gC chimeric protein (SEQ ID NO:22). The sequences upstream and downstream of the HS binding site portion are shown. IL13 was inserted between the two restriction enzyme sites that are underlined. FIG. 2B. The domain of the gB open reading frame (i.e., ORF) from which the poly lysine [poly(K)] sequence was deleted (SEQ ID NO:23). The underlined sequences (codons 68-77) were not present in gB amplified from R5107. FIG. 2C. The amino-terminal sequence of IL13-gD (SEQ ID NO:24). The first underlined sequence identifies the gD signal peptide. IL13 (bracketed by arrows) was inserted between residues 24 and 25 (underlined) of gD, between the XhoI and KpnI restriction enzyme sites.

FIG. 3. Verification of R5111 viral DNA by PCR. Photographs of electrophoretically separated PCR products amplified directly from the plaques picked from Vero (FIG. 3A) and HEp-2 (FIG. 3B) cells. Viral DNAs were extracted as described in Example 1 and subjected to PCR with “IL13” primers from the IL13 ORF and IL13-gD primers, which bracketed IL13 and the gD ectodomain.

FIG. 4. Photograph of electrophoretically separated proteins from lysates of cells infected with R5111 reacted with antibody to gC, gD or IL13. HEp-2 cells grown in 25 cm² flasks were exposed to 10 PFU of HSV-1 or R5111 per cell. The cells were harvested 24 hours after infection, solubilized, subjected to electrophoresis in 10% denaturing polyacrylamide gels, electrically transferred onto a nitrocellulose sheet, and exposed to a monoclonal antibody against gD (FIG. 4A), gC (FIG. 4B) or IL13 (FIG. 4C), respectively. The protein bands corresponding to the gC, IL13-gC fusion protein, gD and the IL13-gD fusion protein are indicated. IL13-gC was the same size as native gC, as expected.

FIG. 5. HA-tagged IL13Rα2 expression from individual clones of stable transfectants of the J1.1 cell line. The individual clones were amplified as described in Example 1. Cells were harvested from 25 cm² flasks, solubilized, and subjected to electrophoresis in 12% denaturing polyacrylamide gels, electrically transferred onto a nitrocellulose sheet, and exposed to a polyclonal antibody to HA tag.

FIG. 6. Diagram of the pgD− vector.

FIG. 7. Schematic representation of the HSV-1 (F) genome and genetic structure of R5141 and R5144.

FIG. 8. Schematic representation of the construction of the ATF-uPA-gD and BD-uPA-gD recombinant viruses. (FIG. 1A) Schematic representation of the uPA constructs. (Line 1) Full length of uPA. (Line 2) ATF-uPA. (Line 3) BD-uPA. (FIG. 1B) Schematic representation of recombinant HSV 5181 and 5182. (Line 1) Sequence arrangement of HSV-1 genome where rectangular boxes represent the inverted repeat sequences ab and b′a′ flanking the unique long (UL) sequence and inverted repeat c‘a’ and ca flanking the unique short (US) sequence. (Line 2) Schematic representation of recombinant HSV-1 (F) genome, in which the N-terminal domain of gC was replaced with IL-13, and the polylysine domain (codons 68-77) of gB was deleted. The domain of gD was replaced with the immediately early promoter of cytomegalovirus to enable the expression of gI. (Line 3) Sequence arrangements of the glycoprotein D are highlighted. IL13 was replaced with ATF-uPA (Line 4) or BD-uPA (Line 5).

FIG. 9. Amino acid sequence alignment of ATF-uPA-gD junction and BD-uPA-gD junction. (FIG. 2A) The amino-terminal sequence of ATF-uPA-gD (SEQ ID NO:45). The first underlined sequence identifies the gD signal peptide. ATF-uPA (bracketed by arrows) was inserted between residues 24 and 25 (underlined) of gD, between the XhoI and KpnI restriction enzyme sites. (FIG. 2B) The amino-terminal sequence of BD-uPA-gD (SEQ ID NO:46). BD-uPA (bracketed by arrows) was inserted between residues 24 and 25 (underlined) of gD, between the XhoI and KpnI restriction enzyme sites.

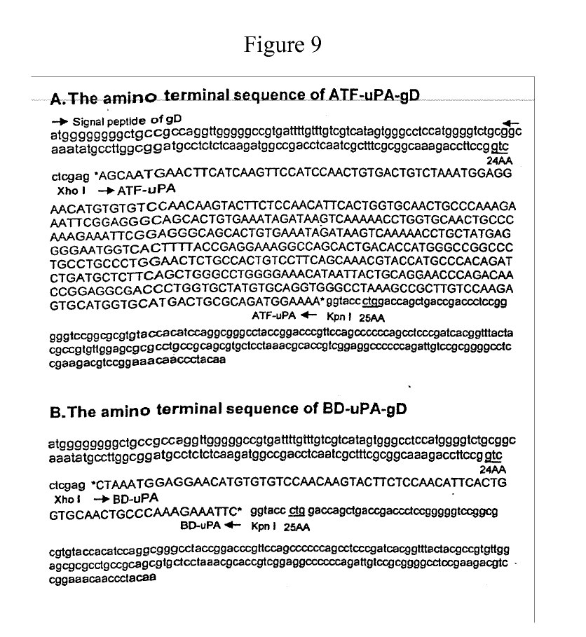

FIG. 10. Photograph of electrophoretically separated proteins from lysates of cells infected with ATF-uPA-gD or BD-uPA-gD virus reacted with antibody to ICP0, gD, US11 or ATF-uPA. Vero cells grown in 25-cm² flasks were exposed to 10 pfu of HSV-1, ATF-uPA-gD or BD-uPA-gD virus per cell. The cells were harvested 24 hours after infection, solubilized, subjected to electrophoresis in 10% denaturing polyacrylamide gels, electrically transferred onto a nitrocellulose sheet, and reacted with a monoclonal antibody against ICP0, gD, US11 or ATF-uPA, respectively. The protein bands corresponding to the ICP0, gD, ATF-uPA-gD fusion protein, BD-uPA-gD fusion protein and US11 are indicated.

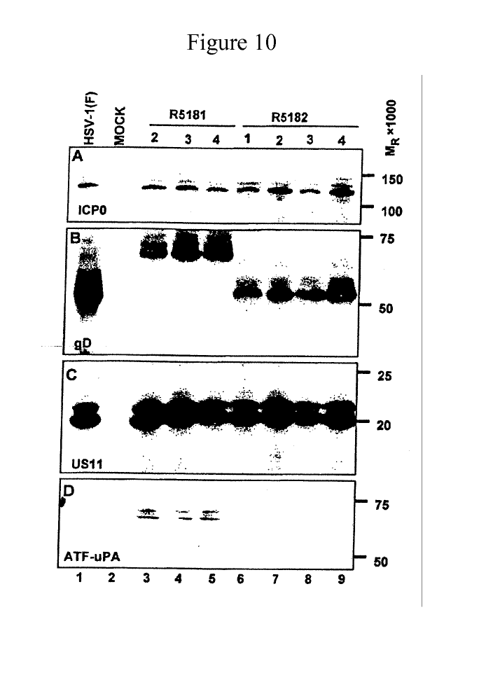

FIG. 11. Human uPAR expression from the individual clones of stable transfectants of the J1.1 cell line. The individual clones were amplified as below. The cells were harvested from 25-cm² flasks, solubilized, and subjected to electrophoresis in 10% denaturing polyacrylamide gels, electrically transferred onto a nitrocellulose sheet, and reacted with a monoclonal antibody to human uPAR.

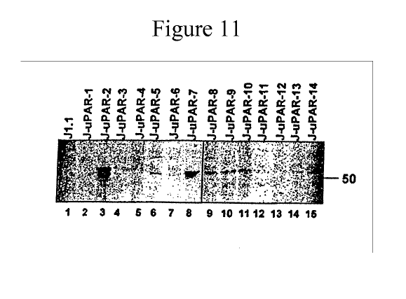

FIG. 12. Replication of R5182 and R5182 virus and HSV-1 (F) in J-uPAR, J1.1, J-HveA and J-Nectin cells. Cells grown in 25-cm² flasks were exposed to 0.1 pfu of the recombinant virus or wild-type HSV per cell and harvested 24 hours after infection. Progeny virus was titered on Vero cells.

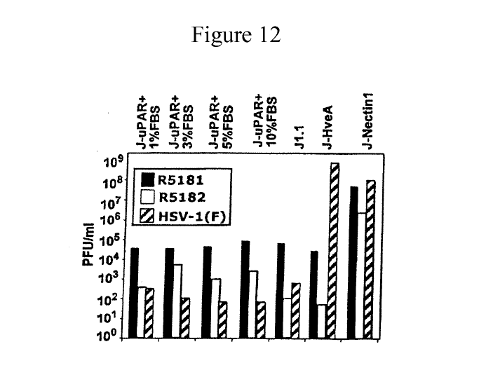

FIG. 13. Photographs of DNA bands derived by reverse transcription and PCR amplification of RNAs extracted from J1.1 or J-uPAR cells using primers as described below. The PCR was performed with primers specific for the hamster (A) and human uPAR (B) ORFs indicated.

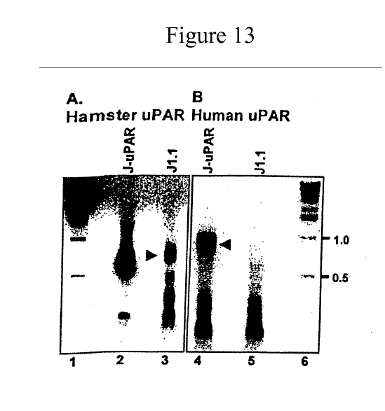

FIG. 14. scuPA competition assay on R5181 virus infectivity in J-uPAR and J1.1. J-uPAR and J1.1 were exposed 1 hour to either 10 nM or 100 nM of scuPA, respectively. The cells were then infected with 0.1 pfu of R5181 virus per cell and harvested 24 hours post-infection. Progeny virus was titrated on Vero cells.

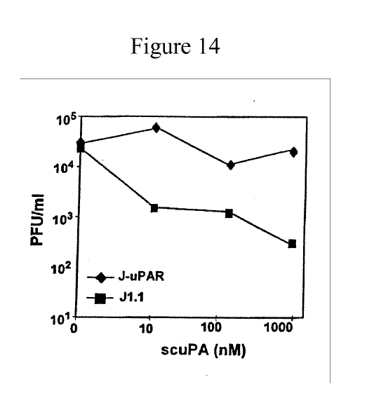

FIG. 15. The Effect of NH₄Cl on R5181 virus infectivity in J-uPAR and J1.1. Cells grown in 25-cm² flasks were exposed to increasing concentrations of NH₄Cl for 30 minutes, infected with R5181 virus at 0.1 pfu/cell for 120 minutes in the same medium, and harvested 24 hours after infection. Progeny virus was titered on Vero cells.

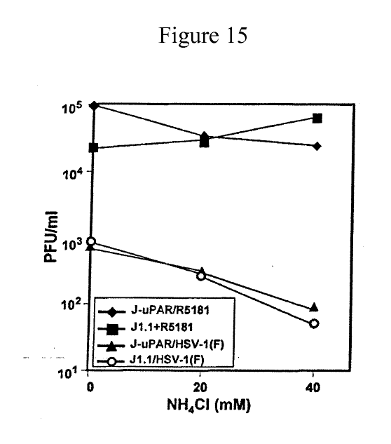

FIG. 16. Amino acid sequence of wild type and R5322 gD

The 155 amino-terminal residues of uPA were inserted between the gD signal peptide and residue 33 of mature gD. (Other positions identified use the numbering of the mature gD.) In addition, valine 34 was substituted with serine. The frameshifts resulting from deletion of a cytosine after codon 60 and insertion of a cytosine after codon 201 introduced in frame new codons including 4 stop codons at the positions shown. The wild-type gD sequences resume at codon 202. Residue 219 is the first methionine of the carboxyl terminal portion of gD.

FIG. 17. Structure of chimeric gDs present in R5322 or designed for construction of recombinants R5321 or R5323 viruses and the properties of the recombinant viruses Panel A, the sequence arrangement of gD present in R5322 or constructed for other recombinant viruses. Panel B, the replication of recombinant viruses in J-Nectin, J-HveA or Vero13R cell lines. Cells grown in 25-cm2 flasks were exposed to 0.1 PFU of the recombinant virus per cell and harvested 24 h after infection. Progeny virus was titrated on Vero-13R cells. Panel C, photograph of electrophoretically separated proteins from lysates of cells infected with R5321, R5322, and R5323 recombinant viruses. Vero-13R cells grown in 25-cm2 flasks were exposed to 1.0 PFU of R5321, R5322, and R5323 virus per cell. The cells were harvested 24 h after infection, solubilized, subjected to electrophoresis in 10% denaturing polyacrylamide gels, electrically transferred onto a nitrocellulose sheet, and reacted with a monoclonal antibody against ICP0, gD(ZC25), uPA and IL13 respectively.

FIG. 18. Structure of chimeric gD and the properties of the recombinant viruses incorporating these glycoproteins. Panel A. sequence arrangement of the chimeric gD designed for construction of recombinant viruses. Panel B. the yields of R5332 and R5352 from cells infected with 0.1 PFU of virus/cell. The recombinant viruses R5331 and R5351 did not infect these cells or produce plaques in VeroIL13 cells. Panel C. accumulation of ICP0. the N-terminal polypeptide of gD identified by the antibody against uPA or the C-terminal polypeptide of gD identified by its reactivity with the anti-Myc antibody.

FIG. 19. Co-precipitation of the polypeptides consisting of the N-terminal uPA-gD33-60 and the C-terminal gD219-314 domains (numbering of mature gD; add 25 for the numbering of SEQ ID NO:26). Panel A: the structure of the plasmids transfected in a pair-wise manner into HEK293 cells. The transfected cells were harvested, 40 hours after transfection. Portions comprising 10% of the total cell lysates were solubilized, subjected to electrophoresis in a denaturing gel and probed with antibody to uPA (Panel B-a, Panel C-a), IL13 (Panel B-a) or gD (Panel B-a) or with antibody to Myc (Panel B-b, Panel C-b). Other aliquots of the lysates were reacted with antibodies to gD, uPA or IL13. The precipitates were collected, solubilized subjected to electrophoresis in denaturing gels and reacted with antibody to uPA (Panel B-c) or Myc (Panel B-d).

### DETAILED DESCRIPTION

The invention provides benefits that will improve the health and well-being of animals such as man by providing a targeted approach to the treatment of a variety of conditions and diseases that currently impair health, resulting in significant economic burdens using conventional treatments. In providing modified viral particles having controllable targeting capacities, the diagnostic and therapeutic benefit of the viruses themselves can be delivered with greater precision to particular cells. Additionally, these viral particles can be used as targeting vehicles for the delivery of a wide variety of therapeutic and diagnostic biomolecules, such as polynucleotides encoding therapeutic or diagnostic peptides.

Beyond the modified viral particles, the invention provides methods for making such therapeutic and diagnostic agents as well as methods for using the agents to diagnose or treat a variety of diseases and conditions, such as tumorigenic disease (e.g., gliomas). To facilitate an understanding of the invention and all of its aspects, illustrative embodiments are described below. The descriptions of these illustrative embodiments are not meant to limit the invention to the embodiments disclosed herein. In light of the description, one of skill in the art will understand that many changes and modifications can be made to the illustrative embodiments and still remain within the invention. The illustrative embodiments are disclosed using as an exemplary virus a member of the Herpesviridae family of viruses, herpes simplex virus (HSV).

As noted above, HSV-1 and HSV-2 are members of the family of viruses known as the Herpesviridae, whose structures are well known in the art. The targeting methods of the invention are applicable to any member of the Herpesviridae and are not limited to the exemplary embodiments described in the examples. Furthermore, a large number of recombinant HSV viruses are known in the art. Such viruses may contain one or more heterologous genes. Also, such viruses may contain one or more mutated HSV genes, for example, mutations that render the virus replication-deficient or affect the virulence of the virus in one or more cell types.

Examples of recombinant HSV containing a heterologous gene and methods of making and using such viruses are described in U.S. Pat. No. 5,599,691 (incorporated herein by reference in its entirety). Preferred heterologous genes include genes encoding marker proteins. Marker proteins, such as green fluorescent protein, luciferase, and beta-galactosidase, allow detection of cells expressing the protein. In other embodiments, the heterologous gene encodes an enzyme that activates a prodrug thereby killing adjacent uninfected cells. In yet other embodiments, the heterologous gene encodes a protein that affects the immune response, such as interleukin 12 (IL-12). Such proteins that activate the immune response against a tumor are particularly useful.

The 369-residue glycoprotein D (gD) is a receptor-binding protein of herpes simplex virus 1 involved in cellular entry; the protein appears to function as a dimer (9). The common receptors for viral entry are Nectin-1, HveA, and a specific O-linked sulfated proteoglycan. The major receptor-binding sites of gD are at the N-terminus whereas the domain required for fusion of the viral envelope with the plasma membrane is at the C-terminus of the ectodomain (residues 260-310). These become actively engaged in membrane fusion following the interaction of the N-terminal domain with one of its cognate receptors (6-8, 10-14).

The domains of gD critical for its functions may be summarized as follows: The interaction of gD with HveA is abolished by deletion of the N-terminal 32 residues of gD. The interactions of gD with Nectin-1 are abolished by mutations at gD residues 34, 38, 215, 222, and 223. A pro-fusion domain, essential for activation of fusogenic glycoproteins gB, gH and gL has been mapped to residues 260 to 310 in close proximity to the transmembrane domain beginning at residue 314 (6). gD suppresses apoptosis induced by mutants lacking gD (17). Linker insertion analyses have identified several sites that abolish this function and some are located between residues 61 to 218 (17). Although failure of many linker insertion mutants to inactivate infectivity was attributed to the flexibility of the gD structure, in view of the disclosure herein, the data can be understood as showing that at least a portion of gD plays no sequence-specific role in virus entry other than to hold the receptor binding domain linked to the pro-fusion domain.

Efforts to re-target gD to the urokinase plasminogen activator receptor for therapeutic applications led to a genetically engineered infectious virus in which the receptor-binding domain, consisting of the N-terminal domain of urokinase plasminogen activator (uPA) fused to residues 33 to 60 of gD, was separated from an independently expressed C-terminal domain of gD containing residues 219 to 369 (numbering of mature gD; add 25 to each number for the numbering used in SEQ ID NO:26). The intervening sequence (residues 62 to 218 of mature gD) was replaced by a stop codon and a promoter for the C-terminal domain of gD. The physical interaction of the two components was reconstructed by co-immune precipitation of the N-terminal domain of uPA with the C-terminal domain of gD. These results indicate that codons 61 to 218 of mature gD do not encode executable functions required for viral entry into cells and suggest that the receptor-binding ligand must interact with, but need not alter the structure of, the residual portion of gD to effect virus entry. This finding opens the way for development of a family of recombinant viruses in which the pro-fusion domain of gD is independently furnished, with receptor-binding domains interacting with the pro-fusion domain to effect entry of the virus via a range of receptors not involved in wild-type HSV entry.

The data disclosed herein establish that there are two domains of gD involved in specific viral entry. An N-terminal domain recognizes specific targets used by wild-type HSV to effect cell entry. That target domain, however, also provides for interaction with a second domain relevant to entry, i.e., a C-terminal fusogenic domain that interacts with fusogenic gB, gH and gL, thereby coordinating the entry process. Constructs disclosed herein provide the N-terminal sub-domain of gD responsible for interaction with the C-terminal fusogenic domain of gD, with and without the N-terminal sub-domain of gD conferring specific target recognition. Thus, constructs provide for an N-terminal target domain of gD that both specifically recognizes a target and that interacts with the C-terminal fusogenic domain of gD. Other constructs provide for an N-terminal sub-domain of gD providing the capacity to interact with the C-terminal fusogenic domain but lack the specific targeting capacity of full-length gD. The invention comprehends the latter constructs as cassettes useful for screening for specific targeting sequences, such as a sequence encoding uPA, as well as products useful in the simplified construction of HSVs designed to specifically recognize any desired target amenable to specific interaction with a peptide.

Consistent with the foregoing principles, the invention comprehends a coding region for a peptide forming a binding pair with a target of choice, that coding region optionally, although typically, being translationally fused to a coding region specifying the N-terminal interaction sub-domain (e.g., amino acids 33-60 of mature gD). In embodiments in which the targeting peptide (e.g., uPA) is capable of directly interacting with a C-terminal fusogenic peptide (e.g., amino acids 219-369 of mature gD), (see the Examples below), the targeting peptide may be encoded by a distinct polynucleotide. The targeting peptide or targeting peptide fusion may be further fused to a peptide comprising the C-terminal fusogenic peptide (e.g., mature gD amino acids 219-369), such as by direct translational fusion to a coding region for residues 219-369 or by translational fusion to a coding region for amino acids 61-369 of mature gD. In additional embodiments, the coding region encodes a portion of the region of mature gD (amino acids 61-218) not essential to viral entry. Additionally, constructs are contemplated that translationally fuse a linker peptide between the targeting peptide fusion coding sequence and the fusogenic peptide coding sequence. Coding regions for any of the peptide linkers known in the art may be used in this context, such as the (Gly₄Ser)ₙ (n=3-5) series of linkers used in engineered antibody products. Use of a linker is expected to provide the spacing and flexibility compatible with effective interaction of the targeting fusion peptide and the fusogenic peptide. Further, constructs embraced by the invention provide a linker interposed between a targeting peptide and a peptide providing the N-terminal sub-domain for interaction with the fusogenic peptide. Any linker known in the art may be used in such constructs to provide the spacing and flexibility compatible with effective target interaction by the targeting peptide with effective interaction between the fusogenic peptide and the N-terminal fusogenic interaction sub-domain of gD.

The targeting peptides contemplated by the invention include any peptide capable of specifically interacting with a target of choice. Typically, the chosen targets will be peptides presented on the surface of a host cell targeted for HSV entry. Non-peptide targets are also comprehended, as are targets not presented on the surface of a host cell, provided that the specific interaction between the target and the HSV targeting fusion leads to viral entry into the host cell (e.g., the targeting fusion may specifically bind a binding pair partner that isn't naturally associated with a host cell but that is capable of interacting specifically with the host cell of interest, effectively “marking” that host cell for viral entry). Beyond naturally occurring members of a binding pair, and fragments and variants thereof retaining the capacity for specific interaction with the binding pair partner, the invention contemplates engineered targeting peptides, such as antibody products, and functional fragments and variants thereof. For example, a single-chain variable fragment of an antibody specifically interacting with a peptide preferentially displayed on the surface of a target host cell, such as a cancer cell, may be fused to the N-terminal sub-domain of gD capable of interaction with the fusogenic domain of gD. In other embodiments, the minimal complementarity determining regions (CDRs) of an antibody are linked in a peptide fused to the interaction sub-domain of gD. Another alternative embodiment places the single variable region, or minimal CDR determinants thereof, of a camelid antibody into a fusion peptide with the interaction sub-domain of gD. In fact, any engineered antibody product, or non-antibody binding peptide, that is amenable to peptide fusion, with or without a linker, is suitable for fusion to the interaction sub-domain of gD in the constructs according to the invention.

The preceding constructs are preferred constructs in that these constructs are re-targeted insofar as the natural targeting provided by the complete N-terminal targeting domain of gD is not present to compete with the targeting provided by the peptide member of a binding pair. In some embodiments of the invention, however, retention of the wild-type HSV targeting provided by the N-terminal domain of gD is acceptable and, in such embodiments, the invention embraces targeting peptide fusions, with or without intervening linkers, to an N-terminal domain of gD extending from a point between amino acids 1-32, inclusive, to amino acid 60 of gD.

The invention also contemplates screens or assays for identifying suitable targeting peptides. In this aspect of the invention, a wide variety of coding regions may be ultimately linked by any manner described above to the coding region for the fusogenic domain of gD. Thus, the set of coding regions may be linked directly to the coding region for the fusogenic domain, or may be linked to the complete or partial intervening sequence of gD that is, in turn, fused to the fusogenic domain, or may be fused to a linker that is in turn fused to the fusogenic domain. The collection of constructs (e.g., library) may then be subjected to any of a variety of well-known assays to identify particular constructs that re-target HSV entry. For example, a functional assay may be employed in which a target host cell is monitored for either cell death attending successful infection or for HSV replication associated with successful infection. Alternative assays are available to detect specific interaction of an HSV fusion peptide (targeting peptide ultimately fused to the interaction sub-domain of gD) with a given target, such as a cell-surface peptide, for example using an immunoassay technique such as ELISA.

In the following examples, it is revealed that residues 61 to 218 of gD do not perform functions required for entry of virus into cells. Two series of recombinant viruses were constructed that target receptors uniquely present on the surface of tumor cells (18-20). In one recombinant (R5111) designed to target the IL13α2 receptor enriched in malignant glioma cells, 140 residues encoding IL13 were inserted between residues 24 and 25 of gD. This recombinant virus can infect cells expressing the IL13α2 receptor but it also retained the capacity to interact with Nectin-1 and HveA receptors (18). In the second recombinant virus (R5141), the IL13 ligand was fused to residue 33 of gD. In addition, valine 34 was replaced with serine. This recombinant entered cells solely via the IL13α2 receptor. IL13 added to the medium blocked entry of the recombinant virus expressing the IL13α2 receptor (20). The second target selected for these studies was urokinase plasminogen activator receptor (uPAR), commonly found on the surface of most cells but highly enriched on the surface of cancer cells (19).

In the first of this series of recombinant viruses, R5181, the N-terminal 155 residues of urokinase plasminogen activator (uPA) were inserted after residue 24 of mature gD. This recombinant virus infected cells expressing Nectin-1, HveA, or even hamster cells that lacked known HSV receptors but were subsequently shown to express hamster-specific uPAR mRNA. In this instance, removal of uPAR by digestion of the glucosyl-inositol anchor with phosphatidyl inositol-specific phospholipase C blocked virus entry (19). The results reported below emerged from an attempt to construct a chimeric gD modeled after the recombinant virus R5141 in that uPA sequences were fused to residue 33 of mature gD and valine 34 was replaced with serine. Although the genetically engineered virus designated R5322 replicated as expected in all cell lines tested, sequencing of the chimeric gD present in the recombinant virus revealed that it contained two frame-shift mutations, one each after residues 60 and 201 (mature gD numbering). The frame shifts introduced 4 stop codons between residues 60 and 201. The first available potential initiator methionine codon was at position 219 of mature gD.

In one aspect, the invention relates to altering the surface of an HSV particle to target the virus to a specific receptor. By creating a fusion protein comprising a portion of gD and a ligand (or binding pair member), the virus is targeted to a cell having a cell surface receptor that binds the ligand (or binding pair member). In preferred embodiments, one or more HSV surface proteins, such as gB (SEQ ID NOs:27 and 28), gC (SEQ ID NOs:29 and 30), or gD (SEQ ID NOs:25 and 26), are altered to reduce binding to natural HSV receptors.

“Alterations” of the surface of an HSV particle or HSV surface protein include insertions, deletions, and/or substitutions of one or more amino acid residues. One type of alteration is an insertion, which involves the incorporation of one or more amino acids into a known peptide, polypeptide or protein structure. For ease of exposition, alterations will be further described using a protein exemplar. Insertions may be located at either or both termini of the protein, or may be positioned within internal regions of known proteins, which yield proteins such as fusion proteins and proteins having amino acid tags or labels.

Another type of alteration is a deletion, wherein one or more amino acid residues in a protein are removed. Deletions can be effected at one or both termini of the protein, or with removal of one or more residues within the amino acid sequence. Deletion alterations, therefore, include all fragments of a protein described herein.

Yet another type of alteration is a substitution, which includes proteins wherein one or more amino acid residues are removed and replaced with alternative residues. In one aspect, the substitutions are conservative in nature; however, the invention embraces substitutions that are also non-conservative. Conservative substitutions for this purpose may be defined as set out in Tables A or B, below.

Amino acids can be classified according to physical properties and contribution to secondary and tertiary protein structure. A conservative substitution is recognized in the art as a substitution of one amino acid for another amino acid that has similar properties. Exemplary conservative substitutions are set out in Table A as described in Lehninger, [Biochemistry, 2nd Edition; Worth Publishers, Inc. New York (1975), pp. 71-77] and set out immediately below.

TABLE A
Conservative Substitutions I

| SIDE CHAIN CHARACTERISTIC | AMINO ACID |
|---|---|
| Non-polar (hydrophobic): | |
| A. Aliphatic | A L I V P |
| B. Aromatic | F W |
| C. Sulfur-containing | M |
| D. Borderline | G |
| Uncharged-polar: | |
| A. Hydroxyl | S T Y |
| B. Amides | N Q |
| C. Sulfhydryl | C |
| D. Borderline | G |
| Positively charged (basic) | K R H |
| Negatively charged (acidic) | D E |

Alternative, exemplary conservative substitutions are set out in Table B, immediately below.

TABLE B
Conservative Substitutions II

| ORIGINAL RESIDUE | EXEMPLARY SUBSTITUTION |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

The binding site of HveA has been reported to be at the amino terminal domain of gD (Carfi A., et al., 2001). The precise binding sites of gD for Nectin 1 are not known, although it has previously been reported to involve gD amino acids 38 and 221 (Manoj S., et al., 2004; Zago A., et al., 2004; Connolly S A., 2005). Accordingly, in one aspect the invention relates to amino acid alterations in the N-terminal region of gD such that the ability of gD to bind HveA or Nectin1 is reduced or eliminated. A “natural receptor” as used herein is a cell surface molecule that interacts with wild-type HSV in the absence of human intervention. For example, gB and gC of HSV-1 interact with heparan sulfate proteoglycans in a natural infection. In preferred embodiments, gB and/or gC are altered to reduce or eliminate binding to heparan sulfate proteoglycans. As another example, gD is known to bind to several receptors, including HveA and Nectin-1, in a natural infection. In preferred embodiments, gD is altered to reduce or eliminate binding to HveA and/or Nectin-1.

Receptor-Ligands

As used herein, “receptor” and “ligand” refer to two members of a specific binding pair and, hence, are binding partners. A receptor is that member of the pair that is found localized on the surface of the cell; the ligand is the member of the pair that is found on the surface of HSV. Thus, in certain embodiments, the “ligand” may actually be what the art recognizes as a receptor outside the context of the invention and the “receptor” may be its respective ligand. More generally, the invention comprehends an HSV exhibiting a member of a binding pair, or a fragment thereof that retains the capacity to specifically bind the other member of the binding pair, on its surface and the other member of that binding pair, or a fragment thereof that retains the capacity to specifically bind its partner, is present on the surface of a target cell.

One advantage of the invention is the ability to tailor HSV to target a specific receptor while maintaining infectivity of the virus. In an exemplary embodiment, an HSV particle contains a fusion protein comprising a portion of gD and the cytokine IL-13. Such a virus is able to infect cells expressing the receptor IL-13Rα2. Because IL-13Rα2 is expressed on the surface of cells of malignant gliomas, HSV containing the gD/IL-13 fusion protein are effectively targeted to such cells. Ligands that bind to receptors which are overexpressed or differentially expressed on either tumor cells or cells associated with tumor growth (e.g., neovasculature) are particularly preferred. Examples include the αᵥβ₃-αᵥβ₅ integrins, which are overexpressed in tumor neovasculature; epidermal growth factor receptor (EGFR), which is overexpressed in head, neck, lung, colon, breast, and brain cancer cells; HER-2/Neu, which is overexpressed in breast cancer cells; MUC-1, which is overexpressed in breast, lung, and pancreas cancer cells; and prostate-specific membrane antigen, which is overexpressed in prostate cancer cells. In certain embodiments, the ligand is a single-chain antibody that binds to its cognate specific binding pair member, herein referred to as a receptor. Single-chain antibodies have been shown to be effective in targeting applications, as evidenced by their ability to target retroviruses to specific receptors.

Essentially any two binding pair members or partners may be used as receptor-ligands in the invention. However, it is contemplated that certain factors, such as the distance from the binding site on the receptor to the membrane, or the conformation of the ligand when fused to gD, may affect the efficiency of recombinant HSV fusion to the cell membrane. Therefore, screens for effective receptor-ligand pairs are contemplated, using no more than routine procedures known in the art. Additional screens, conventional in nature, may be used to optimize constructs. One routine method of screening is to follow the protocol provided in the example for candidate receptor/ligand pairs, using IL-13R/IL-13 as a control receptor/ligand pair.

Alternatively, one may use a membrane fusion assay as described in Turner et al., 1998, incorporated herein by reference in its entirety. In the Turner assay, cells transfected with construct(s) encoding gB, gH, gL, and the gD/ligand fusion protein, and cells expressing the receptor, are co-cultured and the cells are examined for membrane fusion. Membrane fusion between gD/ligand-expressing cells and receptor-expressing cells indicates that the candidate receptor-ligand pair (the ligand being a gD/ligand fusion protein) is functional. Constructs encoding functional gD/ligand proteins can then be used to create recombinant HSV that are targeted to cells expressing the receptor.

Cell Targeting

Evident from the preceding discussion, another aspect of the invention is the targeting of a recombinant HSV to a cell having a specific receptor on its surface. In preferred embodiments, a recombinant HSV is designed to comprise a ligand that interacts with a receptor known to be expressed on a cell of interest. The cell of interest is then infected with recombinant HSV. Such targeting methods may be used for a variety of purposes.

In one aspect, a recombinant HSV is used to introduce a heterologous gene into a cell that expresses the receptor. In preferred embodiments, the cell is not infected by, or is poorly infected by, wild-type HSV. Thus, in certain embodiments, the invention provides a vector for transforming a cell of interest with a heterologous gene.

Further, a cell can be rendered a target of a recombinant HSV of the invention. The cell can be rendered a target by transforming the cell to express one member of a binding pair, e.g., a receptor capable of specifically binding a ligand expressed on a recombinant HSV. For example, as described in Example 2, the J1.1 cell line, which was resistant to infection by a recombinant HSV expressing an IL-13 ligand, was rendered susceptible to infection by transforming the cell line with a vector encoding IL12Rα2 to produce the cell line J13R.

Generally, the targeted HSV according to the invention exhibit one member of a binding pair, with the other member of that pair found on the surface of a target cell. In some embodiments of the invention, targeting is achieved with a ligand-receptor binding pair, with the ligand exhibited on the targeted HSV and the cognate receptor found on the surface of the target cell, as described above. Although the invention comprehends embodiments involving binding pairs that do not exhibit a ligand-receptor relationship (e.g., biotin-avidin) and embodiments in which the receptor is exhibited by the targeted HSV (the “receptor” defined above as a “ligand” using an alternative definition of “ligand”) while the cognate ligand is found on the target cell (the “ligand” defined above as a “receptor” using an alternative definition of “receptor”), embodiments in which the targeted HSV exhibits a ligand and the target cell presents the cognate receptor on its surface is used as an illustrative embodiment to reveal the versatility of the invention and to disclose the full scope thereof. For example, several ligands have been used for receptor-mediated polynucleotide transfer. Some ligands that have been characterized are asialoorosomucoid (ASOR) and transferrin (Wagner et al., Proc. Natl. Acad. Sci. USA, 87(9):3410-3414, 1990). A synthetic neoglycoprotein, which recognizes the same receptor as ASOR, has also been used in a polynucleotide delivery vehicle (Ferkol et al., FASEB J., 7:1081-1091, 1993; Perales et al., Proc. Natl. Acad. Sci., USA 91:4086-4090, 1994) and epidermal growth factor (EGF) has further been used to deliver polynucleotides to squamous carcinoma cells (Myers, EPO 0273085). Each of these specific approaches, and other approaches known in the art to achieve some selectivity in DNA delivery, or targeting, are amenable to use in the compositions and methods of the invention and are contemplated as embodiments of the invention.

For embodiments in which a targeted HSV harboring a coding region, e.g., a therapeutic coding region or gene, is delivered to a target cell, the nucleic acid encoding the therapeutic gene product may ultimately be positioned and expressed at different sites. In certain embodiments, the nucleic acid encoding the therapeutic polynucleotide may be stably integrated into the genome of the cell. This integration may place the gene in its native location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or epitomes encode functions sufficient to permit maintenance and replication independent of or in synchronization with the host cell cycle. How the expression construct is delivered to a cell and where in the cell the nucleic acid remains is dependent on the type of expression construct employed, as would be understood in the art.

It is envisioned that promoters subject to cell cycle regulation will be useful in the present invention. For example, in a bicistronic HSV vector designed to treat a disease, disorder or condition by killing a target cell, use of a strong CMV promoter to drive expression of a first gene, such as p16, that arrests a cell in the GI phase is accompanied by expression of a second gene, such as p53, under the control of a promoter that is active in the GI phase of the cell cycle, thus providing a dual-gene approach to ensure that the target cell undergoes apoptosis. Other promoters, such as those of various cyclins, PCNA, galectin-3, E2F1, p53, BRCA1, and, indeed, any suitable promoter or expression element known in the art, could be used.

In embodiments of the invention designed to treat diseases, disorders, or conditions associated with unwanted or excessive cell proliferation, such as cancer or restenosis, HSV is targeted to proliferating cells thereby killing the cells. Because HSV is lethal to infected cells, expression of a heterologous gene is not required. However, in embodiments wherein the lethality of HSV is attenuated, an HSV harboring a gene that is lethal to the infected cell or that prevents proliferation of the infected cell may be used to target a cell.

Alternatively, HSV targeted to specific surface markers can be used to visualize the distribution of tumor cells in tissues. This diagnostic tool had been unavailable because of the indiscriminate binding of HSV to cells. Modification of HSV by eliminating (ablating) or reducing the indiscriminate binding of HSV to heparan sulfate proteoglycans without deleteriously affecting the capacity of such HSV to replicate in both dividing and non-dividing cells makes possible the use of these modified viral forms to visualize the distribution of tumor cells.

In one preferred method for visualizing the distribution of tumor cells, radioactive visualization is achieved by viral thymidine kinase (TK)-dependent incorporation of a radioactive precursor. Methods of molecular imaging of gene expression are well known in the art. Methods often use highly sensitive detection techniques such as positron emission tomography (PET) or single-photon emission-computed tomography (SPECT). In one embodiment, TK expression is measured using a gancyclovir analog, such as 9-(3-[¹⁸F]fluoro-1-hydroxy-2-propoxy)methylguanine, as the tracer or marker (Vries et al., 2002). For a review of imaging TK gene expression using PET or SPECT, see Sharma et al., 2002 or Vries et al., 2002.

A second preferred imaging method is to fuse a non-critical tegument protein (e.g. U_{S}11, which is present in nearly 2000 copies per virus particle) to a marker protein, such as green fluorescent protein, which is capable of being visualized in vivo. Alternatively, a non-critical protein can be fused to a luciferase and the presence of the luciferase visualized with a luminescent or chromatic luciferase substrate. Although a marker protein can be fused to essentially any viral structural protein, preferred viral proteins include gC, gE, gI, gG, gJ, gK, gN, U_{L}11, U_{L}13, U_{L}14, U_{L}21, U_{L}41, U_{L}35, U_{L}45, U_{L}46, U_{L}47, U_{L}51, U_{L}55, U_{L}56, U_{S}10, and U_{S}11. The marker protein also may be fused to thymidine kinase (Soling et al., 2002).

Library Screening

As noted above, HSV comprising a gD/ligand fusion protein can bind and infect cells expressing a receptor to the ligand. In one embodiment, a cell line expressing a receptor is used in screening for ligands of the receptor. cDNA from a cDNA library is cloned into a vector encoding a portion of gD to produce a gD/cDNA-encoded fusion protein. The resulting vectors are then screened for membrane fusion using the assay of Turner et al. described above or by creating recombinant HSV expressing the gD/cDNA-encoded fusion protein and screening the viruses for the ability to infect receptor-expressing cells. Such methods may be used, e.g., to identify a ligand to an orphan receptor.

In other embodiments, mutations in, or variants of, the receptor or ligand are screened to determine whether the mutants or variants maintain the ability to interact with the respective partner. Such methods may be useful in determining the specific residues important in receptor-ligand interaction.

Therapeutic Methods

Another aspect of the invention is the use of the targeted HSV in therapeutic methods. By altering the cell-binding and infectivity properties of the virus, many routes and methods of administration become viable. For example, non-targeted HSV will bind indiscriminately to a variety of cells. Because of this property, large virus numbers are used and intravenous administration is generally not effective. However, by targeting the virus, one may lower the viral load (i.e., quantity of virus), yet maintain or increase efficacy. Furthermore, the targeted HSV can be administered intravenously and produce therapeutic effects.

Therapeutic methods of the invention include those methods wherein an HSV is targeted to a receptor of a cell that contributes to, or is the basis of, a disease or disorder. These targeted HSV can either exploit the therapeutic properties of HSV itself (e.g., the lethality of HSV to infected cells) or the targeted HSV can serve as a vector for the targeted delivery of at least one therapeutic polynucleotide, such as an expressible polynucleotide comprising a coding region. For example, in methods wherein the targeted HSV contains one or more gene products that render the virus toxic to the cell or that prevent or inhibit cell proliferation, a preferred receptor is overexpressed or selectively expressed on harmful or undesirable cells, such as cancer cells. In other embodiments, the targeted HSV encodes a gene product that provides a desired function or activity in the targeted cell, e.g., when a cell has one or more genetic defects preventing the cell from functioning properly.

Additionally, it is contemplated that a therapeutic polynucleotide (e.g., gene or coding region) of a targeted HSV may be engineered to be under the expression control of a cell- or tissue-specific expression control element, e.g., a promoter. In such embodiments, the targeted HSV provide a further enhancement to the selective treatment of a suitable disorder, disease or condition. The targeted HSV is specific for a binding partner located on the surface of those cells for which treatment is intended, and expression of the therapeutic coding region or gene borne by the targeted HSV is limited to particular cells or tissues.

As HSV has been engineered to overcome the barriers to vector-based therapies, the choice of recombinant polynucleotide to be inserted into the vector has widened to the point where a wide variety of diseases, disorders and conditions are amenable to treatment with targeted HSV. A number of diseases are amenable to polynucleotide-based therapy using HSV (see, e.g., Kennedy, et al. Brain. 120, 1245-1259, 1997, incorporated by reference herein in its entirety). Though most attention has focused on cancers, there has been success in treating Parkinson's disease by expressing tyrosine hydroxylase in striatal cells, thus restoring L-dopa-induced nerve repair following axotomy of the superior cervical ganglion. Injection of a vector expressing nerve growth factor resulted in restored levels of tyrosine hydroxylase. More generally, HSV can now be used in polynucleotide-based therapy to replace missing or defective coding regions in the target cells. In the event of an inherited single-geNe disorder (such as Lesch-Nyhan syndrome) where the complete DNA sequence, cause, and effect of the disorder are known, a single polynucleotide replacement mediated by targeted HSV is appropriate and contemplated. Another strategy amenable to the use of targeted HSV is the enhancement of endogenous expression levels of a gene product, e.g., a growth factor or enzyme. Yet another strategy for using targeted HSV is HSV-directed enzyme pro-drug therapy. The delivery of a drug-sensitivity gene would be beneficial in the treatment of, e.g., a malignant brain tumor, making the tumor more susceptible to conventional anti-cancer agents.

In other embodiments, the targeted HSV of the invention provide for vector-mediated delivery of anti-sense oligodeoxyribonucleotides (oligonucleotides). The oligonucleotides, short segments of DNA (e.g., 2-100 nucleotides in length), are delivered to target cells and therein bind to complementary mRNA, thus blocking the expression of specific genes within the target cells. The encoded protein fail to be synthesized, as the mRNA is not be recognized by the translational components of the cell. In preferred embodiments, a deleterious gene is targeted.

In yet other embodiments, targeted HSV are used to deliver polynucleotides, e.g., DNAs encoding gene products, that can recruit or enhance an immune system response, thereby bringing a subject's or patient's own immune system to bear in the treatment of a disease, disorder or condition known in the art to be amenable to immune system activity. For example, an increase in cellular antigen expression of tumor cells, mediated by delivery of an expressible coding region for the antigen by a targeted HSV, would enhance the immune response and increase the susceptibility of such tumor cells to host cytotoxic immunity.

In some embodiments, a targeted HSV composition of the invention is delivered to a patient at or around the site of a tumor, which is a very efficient method for counteracting clinical disease. Alternatively, systemic delivery of targeted HSV compositions may be appropriate in other circumstances, for example, where extensive metastasis has occurred, or where inaccessible tumors are encountered.

It is contemplated that in certain embodiments of the invention a protein that acts as an angiogenesis inhibitor is targeted to a tumor. Also, an angiogenesis inhibitor agent may be administered in combination with a targeted HSV of the invention. These agents include, for example, Marimastat (British Biotech, Annapolis Md.; indicated for non-small cell lung, small cell lung and breast cancers); AG3340 (Agouron, LaJolla, Calif.; for glioblastoma multiforme); COL-3 (Collagenex, Newtown Pa.; for brain tumors); Neovastat (Aeterna, Quebec, Canada; for kidney and non-small cell lung cancer) BMS-275291 (Bristol-Myers Squibb, Wallingford Conn.; for metastatic non-small cell lung cancer); Thalidomide (Celgen; for melanoma, head and neck cancer, ovarian, and metastatic prostate cancers; Kaposi's sarcoma; recurrent or metastatic colorectal cancer (with adjuvants); gynecologic sarcomas, liver cancer; multiple myeloma; CLL, recurrent or progressive brain cancer, multiple myeloma, and non-small cell lung, nonmetastatic prostate, refractory multiple myeloma, and renal cancer); Squalamine (Magainin Pharmaceuticals Plymouth Meeting, Pa.; non-small cell lung cancer and ovarian cancer); Endostatin (EntreMEd, Rockville, Md.; for solid tumors); SU5416 (Sugen, San Francisco, Calif.; recurrent head and neck, advanced solid tumors, stage IIIB or IV breast cancer; recurrent or progressive brain (pediatric) cancer; ovarian cancer, AML (acute myeloid leukemia); glioma, advanced malignancies, advanced colorectal cancer, von-Hippel Lindau disease, advanced soft tissue cancer; prostate cancer, colorectal cancer, metastatic melanoma, multiple myeloma, malignant mesothelioma: metastatic renal, advanced or recurrent head and neck cancer, metastatic colorectal cancer); SU6668 (Sugen San Francisco, Calif.; advanced tumors); interferon-α; Anti-VEGF antibody (National Cancer Institute, Bethesda Md.; Genentech San Franscisco, Calif., for refractory solid tumors; metastatic renal cell cancer, in untreated advanced colorectal cancer; EMD121974 (Merck KCgaA, Darmstadt, Germany, for HIV-related Kaposi's sarcoma, and progressive or recurrent Anaplastic Glioma); Interleukin 12 (Genetics Institute, Cambridge, Mass., for Kaposi's sarcoma) and IM862 (Cytran, Kirkland, Wash., for ovarian cancer, untreated metastatic cancers of colon and rectal origin, and Kaposi's sarcoma). The parenthetical information following the agents indicates the cancers against which the agents are being used in these trials. It is contemplated that any of these disorders may be treated with the targeted HSV compositions of the invention, either alone or in combination with the agents listed.

In order to prepare a therapeutic composition for clinical use, it will be necessary to prepare the therapeutic composition as a pharmaceutical composition, i.e., in a form appropriate for in vivo applications. Generally, this will entail preparing compositions that are essentially free of pyrogens, as well as other impurities that could be harmful to humans or other vertebrates.

Generally, appropriate salts and buffers are included to render delivery vectors stable and to allow for uptake by target cells. Aqueous compositions of the invention comprise an effective amount of the targeted HSV, dissolved or dispersed in a pharmaceutically acceptable carrier or aqueous medium. Such compositions also are referred to as inocula. The phrase “pharmaceutically acceptable” or “pharmacologically acceptable” refer to molecular entities and compositions that do not produce adverse, allergic, or other untoward reactions when administered to an animal or a human. As used herein, “pharmaceutically acceptable carriers” includes any and all solvents, dispersion media, coatings, antibacterial and antifungal agents, isotonic and absorption delaying agents, and the like. The use of such media and agents for pharmaceutically active substances is well known in the art. Unless a conventional medium or agent is incompatible with either the vectors of the invention or the intended subject receiving treatment, its use in therapeutic compositions is contemplated. Supplementary active or inert ingredients also can be incorporated into the compositions.

The active compositions of the invention include standard pharmaceutical preparations. Administration of these compositions according to the invention is by any known route, provided that the target tissue is accessible via that route. The pharmaceutical compositions may be introduced into the subject by any conventional method, e.g., by intravenous, intradermal, intramusclar, intramammary, intraperitoneal, intrathecal, retrobulbar, intravesicular, intrapulmonary (e.g., term release); sublingual, nasal, anal, vaginal, or transdermal delivery, or by surgical implantation at a particular site. The treatment may consist of a single dose or a plurality of doses over a period of time.

Upon formulation, solutions are administered in a manner compatible with the dosage formulation and in such amount as is therapeutically effective. Appropriate dosages may be ascertained through the use of established routine assays. As studies are conducted, further information will emerge regarding optimal dosage levels and duration of treatment for specific diseases, disorders, and conditions.

In preferred embodiments, the unit dose may be calculated in terms of the dose of viral particles being administered. Viral doses are defined as a particular number of virus particles or plaque forming units (pfu). Particular unit doses include 10³, 10⁴, 10⁵, 10⁶, 10⁷, 10⁸, 10⁹, 10¹⁰, 10¹¹, 10¹², 10¹³ or 10¹⁴ pfu. Particle doses may be somewhat higher (10- to 100-fold) due to the presence of infection-defective particles, which is determinable by routine assays known in the art.

The pharmaceutical compositions and treatment methods of the invention are useful in the fields of human medicine and veterinary medicine. Thus, the subject to be treated may be a vertebrate, e.g., a mammal, preferably human. For veterinary purposes, subjects include, for example, farm animals such as cows, sheep, pigs, horses and goats, companion animals such as dogs and cats, exotic and/or zoo animals, laboratory animals including mice, rats, rabbits, guinea pigs and hamsters; and poultry such as chickens, turkey, ducks and geese.

In some embodiments of the invention, it is contemplated that the targeted HSV is administered in conjunction with chemo- or radiotherapeutic intervention, immunotherapy, or with any other therapy conventionally employed in the treatment of cancer.

To kill cells, inhibit cell growth, inhibit metastasis, inhibit angiogenesis or otherwise reverse or reduce malignant phenotypes using the methods and compositions of the invention, one contacts a “target” cell, a tumor, or its vasculature with a targeted HSV composition and at least one other agent. The components of these compositions are provided in a combined amount effective to kill or inhibit proliferation of cancer cells. This process may involve contacting the cells with the targeted HSV composition and the agent(s) or factor(s) at the same time. This may be achieved by contacting the subject organism, or cell of interest, with a single composition or pharmacological formulation that includes both agents, or by contacting the cell with two distinct compositions or formulations, at the same or different times, wherein one composition includes a targeted HSV composition of the invention and the other composition includes the second agent.

Another aspect of the invention provides diagnostic methods that involve imaging a tumor or diseased tissue using a targeted HSV. Such methods are useful in diagnosing a patient with a disease, disorder, or condition that is indicated by the presence of a receptor on the surface of a cell. Diagnostic imaging methods are discussed above.

Kits

Kits according to the invention may include recombinant viruses of the invention or may include vectors for producing such recombinant viruses. A vector for producing a recombinant virus of the invention may encode the gD/ligand fusion protein or may be designed to facilitate cloning of a ligand to produce a gD/ligand fusion protein (e.g., a vector containing a multiple cloning site within the gD coding region that facilitates in-frame insertions).

Other components that can be included in a kit of the invention include a receptor-expressing cell line (useful as a control), a nucleic acid molecule for expressing the receptor in a particular cell type, and instructions for using the kit to effect diagnostic analyses or therapeutic treatments. In certain embodiments, a therapeutic kit will further contain a component for bringing about a therapeutic effect, such as a prodrug or a toxic compound. In other embodiments, a diagnostic kit will contain a compound useful in imaging methods, such as a chromophore or fluorophore, or an antibody for detecting infected cells.

Having provided a general description of the various aspects of the invention, the following disclosure provides examples illustrative of the invention. In these Examples, the numbering of gD residues is the numbering for mature gD unless indicated otherwise. The complete amino acid sequence of gD provided in SEQ ID NO:26 also includes the N-terminal signal peptide. To convert the numbering for mature gD to SEQ ID NO:26 numbering, add 25, the size of the signal peptide.

### EXAMPLE 1

Construction of HSV Targeting Vector R5111

A targeted HSV was constructed to target the recombinant virus to cells of malignant gliomas. The target for entry of the virus into such cells is the IL13Rα2 receptor known to be present in malignant gliomas. Unlike the more prevalent IL13αR1 receptor, the IL13Rα2 receptor has a shorter cytoplasmic domain and does not interact with IL4, of which IL13 is a close relative. In general, the construction of the targeted HSV involved mutagenizing gB and gC to preclude their interaction with heparan sulfate. Also, IL13 was inserted into gD at amino acid 24 thereby disrupting the gD binding site for HveA. The resulting IL13-gD chimeric virus can use IL13Rα2 for entry into cells carrying that receptor.

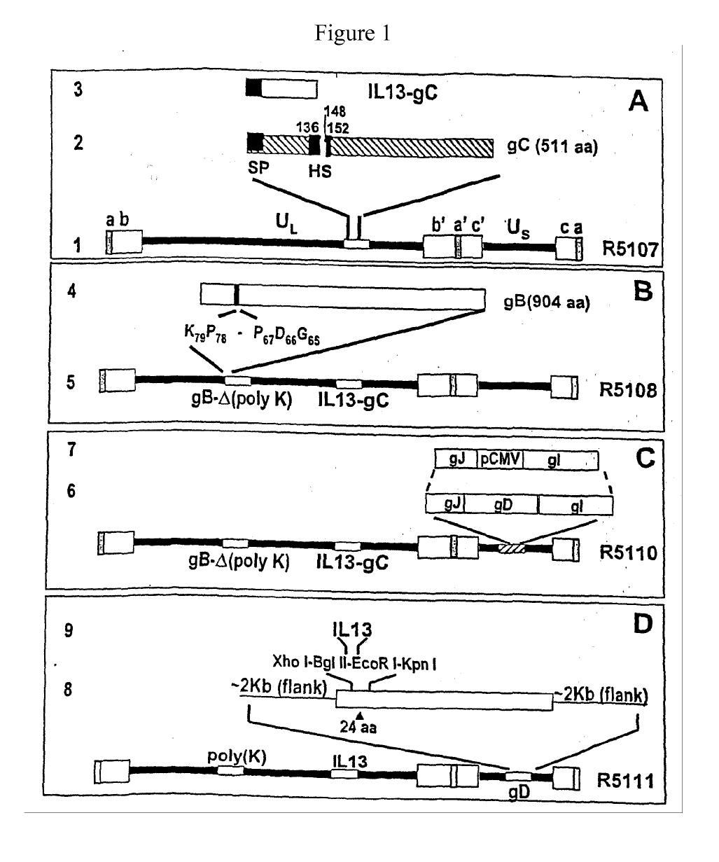

More specifically, the targeted HSV R5111 was constructed in several steps depicted in the four panels of FIG. 1 and detailed below.

(i) Substitution of Amino Terminal Domain of gC with IL13 Fused to the Signal Sequence of gC.

FIG. 1A, lines 1-3 schematically depicts a cDNA consisting of the IL13 coding sequence fused at its amino terminus to its signal sequence. The complete cDNA of IL13, with the N-terminal signal peptide coding region, was amplified using the PCR primer elongation method. The primers were as follows:

pIL13F1,

(SEQ ID NO: 1)

CATTGCTCTCACTTGCCTTGGCGGCTTTGCCTCCCCAGGCCCTGTGC-
CTCCCTCTACAGC;
pIL13F2,

(SEQ ID NO: 2)

GCAGCTAGCCTCATGGCGCTTTTGTTGACCACG-
GTCATTGCTCTCACTTGCCTTGGCGGC;
and
pIL13REcoRI,

(SEQ ID NO: 3)

GAGCTCGGATCCTGAATTCAACCGTCCCTC.

First-round PCR used pIL13F1 and pIL13REcoRI as primers, with pRB5830 (containing the IL13 coding region) as the template. The PCR reaction mixture was then diluted 10-fold and 1 μl of the diluted reaction mixture was used as template for the second round of PCR amplifications with pIL13F2 and pIL13REcoRI as the primer set. The PCR product was gel-purified, digested with NheI/EcoRI, and ligated into pBluescript II KS(+) at XbaI/EcoRI sites to generate pRB5832. To construct the transfer plasmid pRB5835, a 4.8-kbp HindIII/Sac fragment containing the HSV-1 gC coding region was released from cosmid pBC1007 and inserted into pBluescript II KS(+) to generate pRB5833. pRB5833 was cleaved with NheI and EcoRI and the N-terminal 148 residues of gC were replaced with the gC-signal/IL13 chimeric sequence (pRB5834). The insert in pRB5834 was released by XhoI/SacI digestion and ligated into pKO5Y at the same sites to generate pRB5835.

The recombinant virus R5107 (FIG. 1A, line 1) carrying the IL13-gC chimera was generated with the aid of the BAC-HSV system. RR1 competent cells that harbored bacterial artificial chromosome (BAC)-HSV bacmids were transformed with the transfer plasmid pRB5835 by electroporation. After incubation for 1 hour at 30° C. in LB broth, the transformed bacteria were plated on pre-warmed Zeocine (Zeo) plus chloramphenicol (Cm) (20 μg/ml of each) plates and incubated overnight at 43° C. for integration. The next day, six colonies were picked and each was separately diluted in 1 ml LB. Five μl of the diluted bacteria were then plated on Cm/10% sucrose (Suc) plates, and incubated at 30° C. overnight. To further confirm the loss of the replacement vector, 24 Cm/Suc-resistant colonies (four colonies from each plate) were restreaked in duplicate on Cm LB and Zeo LB plates, respectively. The Suc^{r}/Cm^{r}/Zeo^{r} colonies were further screened by PCR (95° C., 4 minutes for cycle 1; then 35 cycles of 94° C., 1 minute; 60° C., 1 minute; and 72° C., 1 minute). The primers were:

| pgC-F, | |
|---|---|
| GACACGGGCTACCCTCACTATCGAGGGC | (SEQ ID NO: 4; from |
| | nt 96158 to 96185 |
| | in HSV-1 strain |
| | 17), |
| and | |
| pgC-R, | |
| GGTGATGTTCGTCAGGACCTCCTCTAGGTC | (SEQ ID NO: 5; from |
| | nt 96859 to 96830 |
| | in HSV-1 strain |
| | 17). |

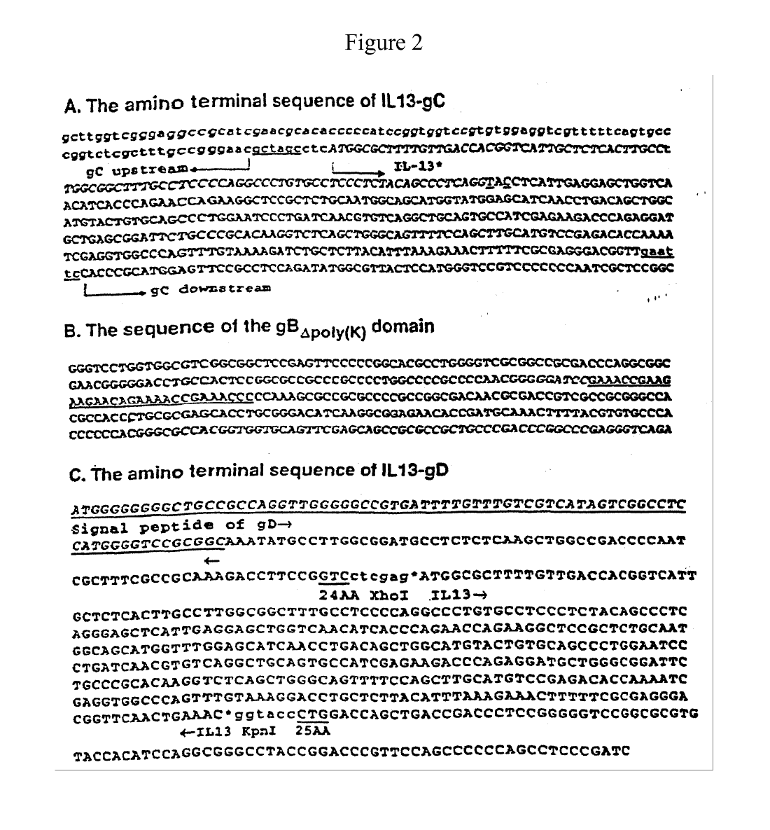

The DNA fragment amplified from PCR-positive clones (FIG. 2B) was sequenced to further confirm the integration of IL13 in the correct open reading frame (ORF) of gC. To verify the viability of the recombinant (R5607), the recombinant BAC-HSV DNA was prepared as described elsewhere (Ye et al., 2000) and transfected into rabbit skin cells by Lipofectamine reagent (Life Technologies, Grand Island, N.Y.). The resultant virus, R5607, was stored at −80° C.

(ii) Deletion of the polylysine track from gB, FIG. 1 panel B. To make a transfer plasmid for the deletion of the gB heparan sulfate binding domain (polylysine), a 4.76 kbp BstEII fragment (from nt 53164 to 57923 of HSV-1) containing the U_{L}27 (gB) ORF released from cosmid BC1014 was blunt-ended and cloned into pBluescript II KS (+) at a SmaI site to generate pRB5846. To construct pRB5847, from which the 10-amino-acid polylysine domain of gB was deleted, two fragments flanking the polylysine domain were amplified by PCR from pRB5846. The primer sets were:

| | |
|---|---|
| pgB1BamHI: | |
| GTTCTTCTTCGGTTTCGGATCCCCCG; | (SEQ ID NO: 6) |
| pgB2BspEI: | |
| CGGCATTTCCGGAATAACGCCCACTC; | (SEQ ID NO: 7) |
| and | |
| pgB3BamHI: | |
| CAGAAAACCGGATCCCCCAAAGCCGCC; | (SEQ ID NO: 8) |
| pgB4BsiWI: | |
| GCCAACACAAACTCGTCGTACGGGTAC. | (SEQ ID NO: 9) |

PCR amplified fragments were then cut with BspEI/BamHI, or BsiWI/BamHI and ligated into pRB5846, which had the 1.2 kbp BsiWI/BspEI fragment already deleted. To generate the transfer plasmid pRB5848, the 4.76 kbp insert in pRB5847 was released by XbaI/EcoRV digestion and ligated into pKO5Y at the sites of XbaI and ScaI. Recombinant HSV-1 virus R5108 is based on R5107 with the additional deletion of the gB heparan sulfate binding domain. It was made by the same procedure as BAC-R5607, except that the transfer plasmid pRB5848 was used instead of BAC-HSV wild-type and pRB5835. The sequence of the mutant gB was verified by sequencing the entire ORF.

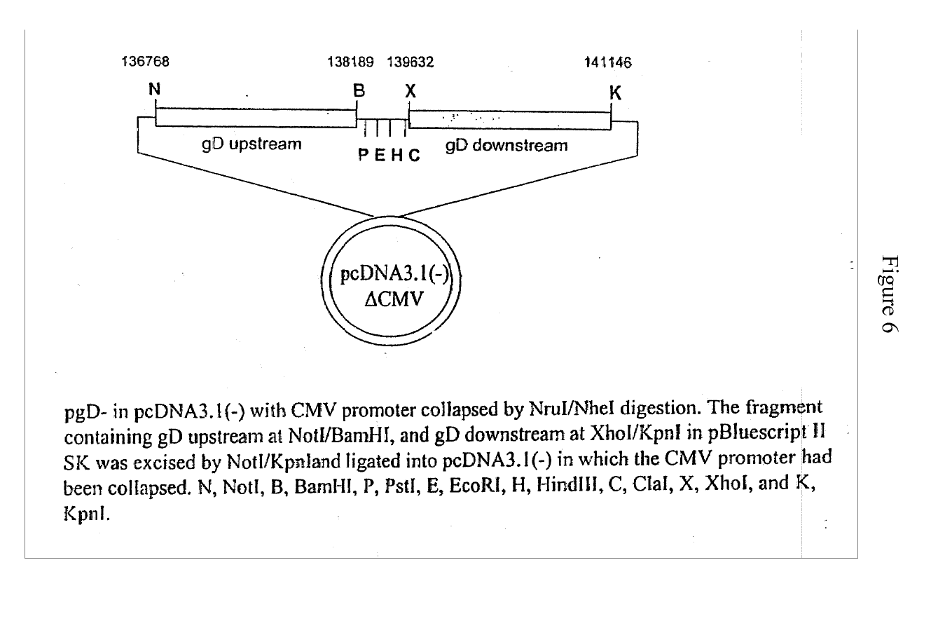

(iii) Deletion of gD (FIG. 1 panel C, lines 6 and 7). The coding sequence of gD was replaced with the human cytomagolovirus immediate early promoter to enable the expression of glycoprotein I. A 0.65 kbp fragment containing the promoter was released from pRB5836 by ClaI digestion and inserted into pgD⁻ (FIG. 6), a plasmid obtained from G. Campadelli-Fiume. This plasmid, containing the flanking sequences of gD but lacking the gD ORF, had been cut with ClaI to generate pRB5849. pRB5849 was then cut with NotI and PmeI and ligated into pKO5Y at the NotI and ScaI sites to generate the transfer plasmid pRB5850. Recombinant HSV-1 virus R5110 is based on R5608 with the additional deletion of gD. It was made by the same procedure as BAC-R5607 except that transfer plasmid pRB5850 was used instead of BAC-HSV wild-type and pRB5835. The recombinant BAC-HSV DNA was prepared as described in (Ye et al., 2000). The mutant virus was designated R5110.

(iv) Construction of the R5111 mutant carrying the IL-13-gD chimeric gene (FIG. 1 panel D). Plasmid pRB123 carries a 6,584 bp BamHI J fragment containing the gD coding region and flanking sequences in the BamHI site of pBR322. To construct the IL 13-gD chimeric plasmid, pRB123 was digested with AflII and HpaI to release two fragments of 7.6 kb and 3.2 kb. The 3.2 kb fragment was further digested with FspI to release 2.5 kb and 0.7 kb fragments that contain the amino-terminal 661 bp of the gD ORF. A polylinker sequence containing the restriction sites XhoI-BglII-EcoRI-KpnI was inserted into the 0.7 kb fragment downstream of the 24th codon of gD by two PCR reactions using a first forward primer,

(SEQ ID NO: 10)

5′-CAGTTATCCTTAAGGTCTCTTTTGTGTGGTG-3′,
and a first reverse primer,

(SEQ ID NO: 11)

5′-CCGGAATTCCGGAGATCTTCCCTCGAGGACCGGAAGGTCTTTGCCGC
GAAAG-3′,
and a second forward primer,

(SEQ ID NO: 12)

5′CCGGAATTCCGGGGTACCCTGGACCAGCTGACCGACCCTCCGG-3′,
and a second reverse primer,

(SEQ ID NO: 13)

5′-CGGGGGGATGCGCAGCGGGAGGGCGTACTTAC-3′,

respectively. After digestion of the two PCR products by EcoRI, they were ligated and amplified by PCR again to obtain the desired DNA fragment containing the polylinker insertion.

IL13 was amplified by PCR with the forward primer,

(SEQ ID NO: 14)

5′-CCGCTCGAGATGGCGCTTTTGTTGACCACGG-3′,
and the reverse primer,

(SEQ ID NO: 15)

5′-GGGGTACCGTTGAACCGTCCCTCGCGAAA-3′,

and then inserted into the XhoI and KpnI sites of the 0.7 kb fragment described above. This new fragment with the IL13 insertion was then ligated with the 2.5 kb and 7.6 kb fragments (see above) to generate the IL13-gD chimeric transfer plasmid, pRB13-24.

R5111 was generated by co-transfection of transfer plasmid pRB13-24 and the R5110 viral DNA into U87 glioma cells. The progeny of the transfection was plated at a high dilution on Vero and HEp-2 cell cultures to yield individual, well-spaced plaques. From each of the infected cell cultures, six single plaques were picked, frozen-thawed, sonicated, and then replated on fresh cultures of Vero or HEp-2 cells (depending on the origin of the plaque) for preparation of virus stocks and to prepare viral DNA for sequencing.

Viral DNA extraction. Infected cells were removed from each of the cm² flasks exposed to individual plaque isolates, rinsed, and resuspended in 500 μl of Lyse-O-Lot (150 mM NaCl, 10 mM Tris, 1.5 mM MgCl₂ in the presence of 0.1% of NP40). Nuclei were removed by low-speed centrifugation. To the supernatant fluid were added sodium dodecyl sulfate (SDS) to 0.2%, EDTA to 5 mM and β-ME to 50 mM. The solution was then extracted twice with phenol/chloroform. Viral DNA was finally precipitated by ethanol, resuspended, and the IL13 ORF and IL13-gD chimeric reading frame were amplified by PCR with two sets of primers. The first set, designed to amplify IL13, consisted of: a forward primer, 5′-CCGCTCGAGATGGCGCTTTTGTTGACCACGG-3′ (SEQ ID NO:16), and a reverse primer, 5′-GGGGTACCGTTGAACCGTCCCTCGCGAAA-3′ (SEQ ID NO:17), which will amplify the IL13 ORF. The second set, designed to amplify the IL13-gD junction, consisted of a forward junction primer,

5′-CCGCTCGAGATGGCGCTTTTGTTGACCACGG-3′ (SEQ ID NO:18), and a reverse junction primer, 5′-AACTGCAGGTTGTTCGGGGTGGCCGGGGG-3′ (SEQ ID NO:19). All 12 IL13-gD PCR products were sequenced to determine whether the gD sequence contained deletions or substitutions.

Verification of the structure of R5111 The construction of the R5111 virus is depicted in FIG. 1. The design involved replacement of the HveA binding site with the IL13 ligand to enable the recombinant virus to bind the IL13α2 receptor on cell surfaces and to delete the sequences reported to bind to heparan sulfate. Verification of the structure of R5111 was done as follows:

(i) The replacement of the amino-terminal domain of gC with IL13 and the disruption of the heparan sulfate binding site was initially verified by sequencing gC from recombinant R5107 (FIG. 2 A).

(ii) The deletion of codons 68-77 of gB was verified by sequencing the gB ORF amplified by PCR from recombinant R5108 (FIG. 2 B). The nucleotide and amino acid sequences of gB with the polylysine track deleted are set out in SEQ ID NOs.:35 and 36, respectively.

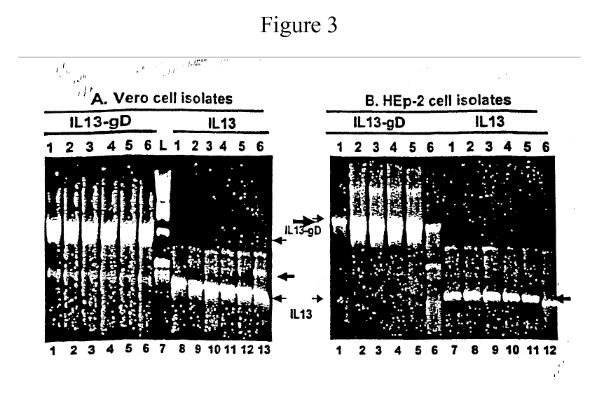

(iii) The presence of chimeric IL13-gD in R5111 was verified by PCR, as illustrated in FIG. 3, and by sequencing the entire IL13-gD coding region, amplified by PCR, as shown in FIG. 2. The nucleotide and amino acid sequences of gD with the IL13 integration are set out in SEQ ID NOs.:39 and 40, respectively. The R5111 recombinant was initially isolated from transfected U87 cells and grown in parallel in Vero cells and HEp-2 cells. To determine whether the virus grown in Vero cells or HEp-2 cells differed with respect to amino acid sequence, six plaques each from Vero or HEp-2 cultures containing well-separated plaques were picked. In this series of verification experiments, two sets of primers were used to confirm the presence of the IL13 insert in gD and to verify the presence of a junction between IL13 and gD. In a second round of verifications, the 12 clones of gD were sequenced to determine whether the isolates obtained from the viruses passaged in Vero cells or in HEp-2 cells differed in amino acid sequence. No differences were found. Furthermore, except for the inserted IL13 sequence, no differences were found between the sequence of HSV-1 (F) gD and those of the cloned IL-13-gD chimeric genes (FIG. 2 C).

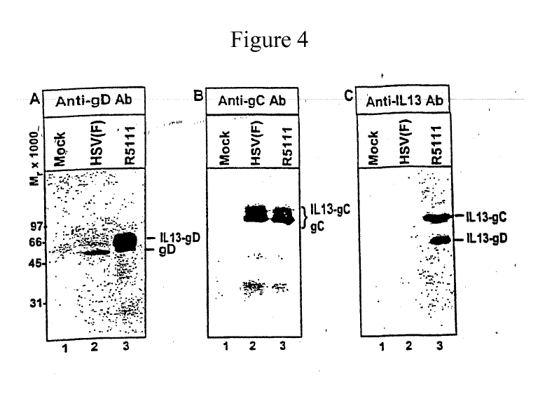

(iv) In denaturing polyacrylamide gels, IL13 migrated as a protein with an apparent Mr of 15-17,000. In the recombinant R5111, IL13 replaced 148 amino acids of gC. FIG. 4B shows an immunoblot of electrophoretically separated proteins from a lysate of R5111 mutant-infected cells exposed to an antibody to gC. As illustrated in that figure, the anti-gC antibody reacted with proteins present in lysates of HSV-1 (F) and with proteins from R5111 lysates, exhibiting similar electrophoretic mobilities. In contrast, an antibody to IL13 reacted with a band of similar mobility in R5111 lysates (FIG. 4C, lane 3) but not in lysates of HSV-1(F) (FIG. 4 C, lane 2). The IL13-gD fusion protein in the R5111 mutant virus was verified by reacting the cell lysates with gD and IL13 antibody. Comparison of wild-type gD and the chimeric IL13-gD chimeric protein (FIG. 4 A, lane 3), showed that, as expected, IL13-gD migrated more slowly than the wild-type gD (FIG. 4 A, lane 2). The faster migrating band of gD did not react with the antibody to IL13 (FIG. 4C, lane 2).

### EXAMPLE 2

Construction of a Cell Line Expressing the IL13 Receptor (IL13Rα2)

A rigorous test of the ability of R5111 to utilize the IL13Rα2 protein as a receptor for entry required construction of a cell line expressing IL13Rα2 (nucleotides 126-1265 of SEQ ID NO.:33; SEQ ID NO:34) in the absence of other HSV-1 entry receptors. The J1.1 cell line was selected for this construction. In essence, this cell line lacks the receptors necessary for the entry of virus into cells and the cell line is not susceptible to infection by wild-type virus. The construction of a plasmid encoding a IL13Rα2 protein fused at its carboxyl terminus to a HA tag, transfection of J1.1 cells with the plasmid encoding the tagged IL13Rα2 protein, and selection of the cell line expressing the protein is described below.

To test for the production of IL113Rα2 protein, five clones of the selected cells were harvested, solubilized, subjected to electrophoresis in denaturing polyacrylamide gels and tested for expression of the protein.

Construction of J13R, a cell line stably expressing IL13Rα2 receptor. The IL13α2 coding region was tagged with an HA tag at its 3′ end (the carboxyl terminus of the encoded polypeptide) by PCR with forward primer, 5′-AAGATTTGGGC-TAGCATGGCTTTCGTTTGC-3′ (SEQ ID NO:20), and reverse primer,

5′-TCCCTCGAAGCTTCAAGCATAATCTGGCACATCATATGTATCACAGAA-AAA-3′ (SEQ ID NO:21). NheI and HindIII restriction digests were used to create compatible ends. The DNA fragment was then inserted into pcDNA 3.1 (zeo) vector (Invitrogen; Carlsbad, Calif.) to generate transfer plasmid pRB 13-R2. All of the constructs were sequenced to insure fidelity.

J1.1, a derivative of BHK thymidine kinase⁻ cells which lack both HveA and nectin 1 receptors, was obtained from Dr. G. Campadelli-Fiume, University of Bologna, Italy. J1.1 cells, stably transfected with pRB 13-R2 using a Lipofectamine kit (Gibco-BRL), were selected on the basis of their resistance to zeocin (Invitrogen). Zeocin-resistant clones were amplified and screened for IL13Rα2 expression by immunoblotting with anti-HA polyclonal antibody. Lysates of parental and transformed cells formed by solubilized in SDS were each electrophoretically separated in a denaturing gel (50 μg/lane), transferred to a nitrocellulose sheet, and probed with antibody against HA (Santa Cruz Biotechnology). The protein bands were visualized by an enhanced chemiluminescent detection (ECL) system (Pierce, Rockford, Ill.) according to the instructions of the manufacturer. One (J13R-2) was selected for testing the ability of R5111 to use the IL13Rα2 receptor.

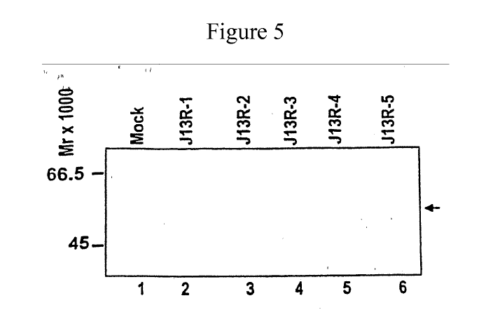

As shown in FIG. 5, all clones expressed a protein band reactive with the anti-HA antibody. The apparent size of the protein was consistent with the reported size of IL13Rα2. Of 5 J13R-positive clones, J13R-2 (FIG. 5, lane 3) was selected and designated J13R.

### EXAMPLE 3

Infection by the HSV Targeting Vector R5111

SK-N-SH, HEp-2, Vero, and U87 cells were obtained from American Type Culture Collection (Rockville, Md.) and maintained in Dulbecco's modification of Eagle's Minimal Essential Medium (DMEM) supplemented with 10% fetal bovine serum. Replicate cultures of SK-N-SH, HEp-2, Vero, U87, J1.1, and J13R were exposed to 0.01 PFU of R5111 virus per cell. After 24 hours of incubation, the cells were harvested and viral yields were titered on Vero cells.

Immunoblotting electrophoretically separated proteins. The indicated cells were mock-infected or exposed to 10 PFU of recombinant or wild-type HSV-1(F) per cell. The cells were harvested at 24 hours after infection, disrupted in SDS disruption buffer, boiled, cleared by centrifugation and electrophoretically separated on a 10% denaturing polyacrylamide gel. After transfer to a nitrocellulose membrane, the isolated proteins were reacted with antibodies as indicated using known and conventional techniques. Monoclonal antibodies against gD− (clone H170), gC- and HA-specific polyclonal antisera were purchased from the Goodwin Institute, Plantation, Fla. Polyclonal antibodies against IL13 were purchased from Santa Cruz Biotechnology.

The results shown in Table 1 were as follows: R5111 replicated to within a 10-fold range in HEp-2, Vero, U87, and J13R cells. The titer obtained from J1.1 cells was approximately 10⁵-fold lower than that obtained from all other cell lines. To test whether the J13R cell line had acquired a receptor for wild-type HSV-1 (HSV-1(F)), J1.1 and J13R cells were also exposed to the wild-type virus. The results, also shown in Table 1, indicate that the cells remain resistant to the wild-type virus. It was known that HEp-2 cells express the nectin receptor but not the HveA receptor. The results show that the targeted HSV containing an IL13-gD fusion can target (i.e., bind and infect) cells expressing a particular receptor (IL13Rα2) approximately as well as wild-type HSV targets cells expressing the HveA receptor. The results indicate that R5111 can use IL13Rα2 as a receptor for entry in a cell line lacking all other HSV-1 receptors.

TABLE 1
Replication of R5111 in various cell lines

| Virus | Cell Line* | Yield** |
|---|---|---|
| R5111 | Vero | 11 × 10⁷ |
| | HEp-2 | 1.2 × 10⁷ |
| | SK-N-SH | 17 × 10⁷ |
| | U87 | 27 × 10⁷ |
| | J1.1 | 2 × 10² |
| | J13R | 11 × 10⁷ |
| HSV-1 (F) | J1.1 | 6 × 10³ |
| | J13R | 4 × 10³ |

*cell lines derived from human brain tumors.
**The cells were exposed to 0.01 PFU of R5111 or HSV-1 (F) per cell and harvested 24 hours after infection. Progeny virus were titered on Vero cells.

This disclosure contains an exemplary description of the construction and properties of a recombinant HSV virus, R5111. In R5111, the heparan sulfate binding sites on the surface of the viral particle were ablated to preclude or at least reduce the attachment of virus to non-targeted cells. Attachment even in the absence of fusogenic activity may lead to endocytosis, degradation of the virus particle, and to potential damage to the cell by lysosomal enzymes (Zhou et al. 2002; Zhou et al. 2000). At the same time, a copy of IL13 was inserted into gC to enhance binding of virus particles to the IL13Rα2 receptor. The major restructuring of the viral genome consisted of insertion of IL13 at amino acid 24 of gD. Available data indicate that this modification ablates the gD binding site for the HveA receptor (Carfi et al. 2001). The data obtained using R5111 indicate that the virus retains the capacity to interact with the Nectin receptor. Nonetheless, the R5111-targeted HSV was able to infect and replicate in J13R cells but not in the parental, J1.1, cells.

### EXAMPLE 4

Construction of HSV Targeting Vector R5141 and R5144

A therapeutic herpes simplex virus 1 (HSV-1) capable of infecting and replicating solely in cells harboring the IL13Rα2 receptor was constructed using recombinant DNA techniques. As disclosed above, construction of R5111, which expresses IL13 on its surface and lacks the binding sites for heparin sulfate, allowed R5111 to infect J-13R cells as well as cells exhibiting the natural receptors for HSV-1. Thus, the involvement of fusogenic glycoproteins of R5111— a key step in viral entry—is independent of the receptor with which gD interacts.

The binding site of HveA has been reported to be at the amino terminal domain of gD (Carfi A., et al., 2001). The precise binding sites of gD for Nectin 1 are not known, although it has previously been reported to involve gD amino acids 38 and 221 (Manoj S., et al., 2004; Zago A., et al., 2004; Connolly S A., 2005). The general assumption within the field is that the HveA and Nectin 1 sites do not overlap and that each independently promotes the same structural alteration of gD to enable entry of the virus into cells. However, it is possible that the surface structure of HveA and Nectin 1 at their binding sites may be similar even though the Hve1 and Nectin1 amino acid sequences are not identical.

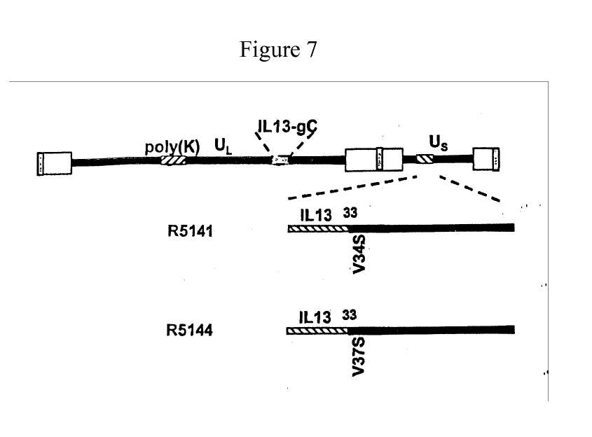

Based in part on the foregoing information, viruses capable of productive replication solely in targeted cells were designed as shown in FIG. 7. Using standard molecular biological cloning techniques known in the art, recombinant virus R5141 was constructed by inserting IL13 in the place of gD residues 1-32. In addition, the valine residue at position 34 was substituted with serine (“V34S”) (SEQ ID NOs.:41 and 42, respectively). Similarly, recombinant virus R5144 was constructed by inserting IL13 in the place of gD residues 1-32, and the valine at position 37 was substituted with serine (“V37S) (SEQ ID NOs.:43 and 44, respectively).

One of skill would appreciate that a variety of re-targeted SHV, both HSV-1 and HSV-2, could be constructed and assessed using routine techniques in view of the disclosures herein. In particular, substituting a binding domain of a binding partner for the N-terminal region of gD, e.g., for amino acids 1-32 of gD, would be within the skill in the art. Further alteration of the gD fusion, e.g., by amino acid substitution, whether conservative substitution or not, would also be within the skill in the art. Of course, additions or deletions to gD fusions would also be within the skill in the art and assessing the targeting capacities of such constructs would involve routine experimentation in view of the teachings herein.

### EXAMPLE 5

Infection by the HSV Targeting Vectors R5141 and R5144

The capacities of the recombinant viruses described in Example 4 to productively replicate solely in targeted cells were assessed using the cell lines which express either HveA (J-HveA) alone (relative to the group of HveA, Nectin1, and IL13R α2), Nectin1 (J-Nectin1) alone, or IL13Rα2 (J-13R) alone. For cell infection, the procedure set forth in Example 3 was repeated using recombinant virus R5141 and recombinant virus R5144.

The replication of R5141 and R5144 in J-Nectin-, J-HveA-, and J-13R-specific cells are summarized in Table 2. R5141 and R5144 do not productively interact with either native gD receptors, HveA or Nectin1. Significantly, however, R5141 interacts with and replicates in IL13Rα2 for productive entry into cells.

TABLE 2
Replication of R5141 and R5144 in J-Nectin, J-HveA and J-13R cells.

| | HSV-1 (F) | R5141 | R5144 |
|---|---|---|---|
| J-Nectin | 4 × 10⁸ | 7 × 10¹ | 5 × 10¹ |
| J-HveA | 3 × 10⁸ | 4 × 10¹ | 3 × 10² |
| J-13R | 3 × 10¹ | 5 × 10⁶ | 7 × 10² |

Thus, recombinant virus R5141 is capable of productive replication solely in targeted cells and this result opens the way for development of therapeutic viruses targeting cells exhibiting the IL13Rα2 receptor, such as malignant gliomas and other human tumors exhibiting IL13Rα2. It is expected that other mutations (i.e., those that abolish binding of Nectin and those that have a similar effect on HveA) will yield viruses that enter solely via non-natural HSV receptors.

### EXAMPLE 6

HSV Targeting Vector R5161

An HSV targeting vector designated HSV R5161 has a structure analogous to the structure of HSV R5141, and HSV R5161 was constructed in the manner described in Example 4, above, with the exception that HSV R5161 contains the sequence encoding the HSV gD leader sequence, whereas HSV R5141 contains the sequence encoding the IL-13 leader sequence. In particular, HSV R5161 encodes an IL-13-gD fusion protein in which IL-13 sequence replaces the sequence encoding gD amino acids 1-32, with a V34S substitution in the gD moiety of the fusion protein, as described above in the context of describing HSV R5141.

The relative capacities of recombinant viruses HSV R5141 and HSV R5161 to productively replicate in targeted cells were measured in the J-13R cell line, which expresses IL13Rα2, but not HveA or Nectin1. HSV R5161 was expressed at a level 10-fold higher than the expression level of HSV R5141 in J-13R cells.

It is expected that in the majority of embodiments of the invention, relatively high levels of expression will be advantageous in ensuring that targeted cells are efficiently contacted by the re-targeted HSV. In re-targeting the HSV, the invention provides an approach to controlling the virulence of the virus in a manner that minimizes undesirable pathogenicity, i.e., pathogenicity towards non-targeted cells. In addition, the virulence of any re-targeted HSV can be further attenuated using known approaches to virulence control that do not interfere with the re-targeting, such as by mutating the γ₁34.5 gene(s). In those embodiments in which it is desirable to have relatively high expression levels of the re-targeted HSV, it is preferred that the leader sequence of gD be used. Alternative leader sequences, such as leaders from other HSV genes, are contemplated. Moreover, expression control elements (e.g., promoters, enhancers, expression factor binding sites) can be engineered to achieve desired expression levels of the fusion protein using ordinary levels of skill and techniques known in the art.

### EXAMPLE 7

Construction of HSV Recombinant Viruses R5181 (ATF-uPA-gD) and R5182 (BD-uPA-gD)

I. Construction of the ATF-uPA-gD Plasmids.

The complete cDNA of amino-terminal fragment (ATF) of uPA without the N-terminal signal peptide was amplified from pULscuPA plasmid using the following primers:

| | |
|---|---|
| ATF-uPA-XhoI: | |
| 5′-CCGCTCGAAGCAATGAACTTCATCAAGT- | (SEQ ID NO: 47) |
| TCCATC-3′ | |
| ATF-uPA-KpnI: | |
| 5′-GGGGTACCTTTTCCATCTGCGCAGTCAT- | (SEQ ID NO: 48) |
| GCACC-3′ | |

The PCR product was gel purified and ligated into pGEM-T Easy Vector (Promega, Madison, Wis.). The sequence of ATF-uPA was verified by sequencing the entire ATF-uPA. Plasmid pGG5112 carries a 3648 bp fragment containing gD, mutant IL-13 (E13Y) and flanking sequences in the EcoRI/XbaI sites of pBR322 (Reuning U, et al., 1998). To construct the ATF-uPA-gD chimeric plasmid, pGG5112 was digested with XhoI and KpnI to release two fragments, 6.2 and 0.4 kb, respectively. Plasmid ATF-uPA was digested with XhoI and KpnI and then inserted into the XhoI and KpnI sites of the 6.2-kb fragment to generate the ATF-uPA-gD chimeric transfer plasmid.

II. Construction of the R5181 (ATF-uPA-gD) and R5182 (BD-uPA-gD) Recombinant Viruses.

The R5181 and R5182 viruses were generated by co-transfection of transfer plasmid ATF-uPA-gD or BD-uPA-gD and the R5110 viral DNA into rabbit skin cells by Lipofectamine reagent (Life Technologies, Grand Island, N.Y.). The R5110 vector was described in WO 2004/033639 A3, incorporated by reference herein; R5110 contains a deletion of gD, a deletion of the heparan sulfate binding domain of gB, and a substitution of the amino terminal domain of gC with IL13 (FIG. 1B).

The progeny of the transfection was plated at a high dilution on Vero cell cultures so as to yield individual, well-spaced plaques. From each of the infected cell cultures, four single plaques were picked, frozen-thawed, sonicated, and then replated on fresh cultures of Vero cells for preparation of virus stocks and viral DNA for sequencing. Vero cells were obtained from the American Type Culture Collection (Rockville, Md.) and maintained in Dulbecco's modification of Eagles minimal essential medium (DMEM) supplemented with 5% newborn calf serum (NBCS). Rabbit skin cells were maintained in DMEM supplemented with 5% NBCS.

III. Viral DNA Extraction.

Infected cells were removed from each of the 25-cm² flasks exposed to the individual plaque isolates, rinsed, and resuspended in 500 μl of Lyse-O-Lot (150 mM NaCl/10 mM Tris/1.5 mM MgCl₂) in the presence of 0.1% of Nonidet P-40 (Zhou et al., 2002). The nuclei were removed by low-speed centrifugation. SDS to 0.2%, EDTA to 5 mM, and β-ME to 50 mM were added to the supernatant fluid, which was then extracted twice with phenol/chloroform. Viral DNA was finally precipitated by ethanol and resuspended, and the ATF-uPA-gD or BD-uPA-gD chimeric reading frames were amplified by PCR with the following primers:

(SEQ ID NO: 49)

5′-CGGAATTCGATGGGGGGGGCTGCCGCCAG-3′

(SEQ ID NO: 50)

5′-AACTGCAGCTAGTAAAACAAGGGCTGGTGCG-3′

The gD PCR products were sequenced to determine whether the gD and ATF-uPA, or BD-uPA sequences contained deletions or substitutions. The R5181 virus encodes an uPA peptide insert of 135 residues in length (residues 20-155 of uPA of SEQ ID NO:52, encoded, e.g., by SEQ ID NO:51) between residues 24 and 25 of the HSV-1 gD of SEQ ID NO:26. The complete polynucleotide and amino acid sequences of ATF-uPA-gD are set out in SEQ ID NOs:53 and 54. R5182 contains a much smaller peptide insert of 23 residues (residues 34-57 of uPA of SEQ ID NO:52) and consisting of the binding site of uPA for uPAR. The complete polynucleotide and amino acid sequences of BD-uPA-gD are set out in SEQ ID NOs:55 and 56. The structures of R5181 and R5182 viruses were verified as follows:

(i) The presence of a chimeric ATF-uPA-gD gene in the R5181 virus and of a BD-uPA-gD gene in R5182 were verified by sequencing of the entire open reading frames amplified by PCR (FIG. 2A and FIG. 2B, respectively).

(ii) As expected, anti-gD antibody reacted with the ATF-uPA-gD band in lysates of cells infected with the R5181 mutant virus but not with the gD band in lysates of wild-type virus (FIG. 3). Moreover, as expected, the chimeric ATF-uPA-gD protein of the R5181 virus (FIG. 3, lanes 3-5), migrated more slowly than wild-type gD (FIG. 3, lane 1).

### EXAMPLE 8

Construction of a Cell Line Expressing uPAR

I. Construction of J-uPAR, a Cell Line Stably Expressing Human uPAR.

To determine whether R5181 and R5182 viruses were able to use the human uPAR protein as a receptor for entry, we constructed, in parallel, a cell line expressing this protein in the absence of other HSV-1 entry receptors. J1.1 cells stably transfected with the human uPAR expression plasmids by using Lipofectamine kit (GIBCO/BRL) were selected on the basis of their resistance to hygromycin B (Invitrogen, Carlsbad, Calif.). Hygromycin B-resistant clones were amplified and screened for uPAR expression by immunoblotting with monoclonal anti-human uPAR antibody (R&D Systems, Inc., Minneapolis, Minn.).

Parental and transformed cells were solubilized in SDS, electrophoretically separated in a denaturing gel (100 μg/lane), transferred to a nitrocellulose sheet, and probed with antibody against uPAR followed by the appropriate secondary antibody conjugated to alkaline phosphatase (Bio-Rad). The protein bands were visualized with 5-bromo-4-chloro-3-indolylphosphate/nitroblue tetrazolium (Denville Scientific, Metuchen, N.J.). As shown in FIG. 4, all clones expressed a protein reactive with the anti-human uPAR antibody but differed in the level of expression. The apparent size of the protein was consistent with the reported size of uPAR. Of 14 J-uPAR-positive clones, J-uPAR-7 (FIG. 4, lane 8) was selected for further studies because it provided the highest expression level of uPAR.

II. Cell surfaces Immunostaining.

Hygromycin B-resistant cells (J-uPAR) in 25-cm² flasks were removed from the dish by gentle scraping and reacted with human uPAR antibodies (5 μg/ml) for 15 minutes on ice. The cells were then rinsed with cold PBS and reacted for 15 minutes with a 1:64 dilution of a goat anti-mouse immunoglobulin G conjugated to fluorescein isothiocyanate (FITC) (Sigma, St, Louis, Mo.) in ice-cold PBS. After rinsing with cold PBS, the cells were suspended in 100 μl of ice-cold PBS for further immunofluorescence analyses. 10 μl of the surface-stained cells were plated onto four-well glass slides and mounted in 90% glycerol. Slides were analyzed with the aid of a Zeiss confocal microscope. A total of 200 cells in adjacent fields were examined for surface fluorescence.

As noted above, uPAR is anchored to the plasma membrane via glycosylphosphatidylinositol and lacks transmembrane and cytosolic domains. The expression of uPAR was investigated in J-uPAR-7 cells using immunofluorescence as the detection method for uPAR. In a first series of experiments, 70% of the J-uPAR-7 cells stained positive for uPAR. (FIG. 4, lane 8) in cultures passaged by gentle scraping of the cells. The number of positive cells after single detachment of cells with versene and totally disappeared after detachment with trypsin. These results indicated that treatment with trypsin or versene irreversibly altered the properties of uPAR.

In a second series of experiments, the effect of fetal bovine serum (FBS) on the expression of uPAR was investigated because FBS could internalize the cell surface receptor. J-uPAR cells were grown in serum-free and specialty medium (AIM-V Medium, Invitrogen, Carlsbad, Calif.) containing different concentrations of FBS. FBS affected the signal intensity but not the percentage of positive cells. Cells grown in AIM-V medium in the absence of serum grew poorly. Moreover, the cells could not be passaged. The results indicated that although FBS negatively affected the presence of uPAR on the cell surface, at least a low concentration of FBS was needed to maintain J-uPAR cells. It is expected that culturing J-uPAR cells, or equivalent cells, in one of the relatively new media formulations known in the art to reduce cell dependence on sera for growth will allow greater expression and cell-surface presentation of uPAR.

### EXAMPLE 9

Infection by the HSV Targeting Vector R5181 and R5182

I. Virus Titration.

Replicate cultures of J-uPAR, J1.1, J-HveA (expresses HveA alone) and J-Nectin-1 (expressed Nectin-1 alone) were exposed to 0.1 pfu of ATF-uPA-gD, BD-uPA-gD, or HSV-1 (F) virus per cell. After 24 hours of incubation, the cells were harvested, sonicated and titrated on Vero cells.

For controls, the indicated cells were mock-infected or exposed to 10 pfu of recombinant or wild-type HSV-1(F) per cell. The cells were harvested 24 hours after infection, disrupted in SDS disruption buffer, boiled, cleared by centrifugation, electrophoretically separated on a 10% denaturing polyacrylamide gel, transferred onto a nitrocellulose membrane, and exposed to appropriate antibodies under standard conditions. The results shown in FIG. 5 are as follows:

(i) HSV-1(F) replicated poorly in J-uPAR or J1.1. The amounts recovered from infected cells may represent, in large part, attached unpenetrated virus.

(ii) The titer obtained from cells infected with R5181 virus from infected J-uPAR cells was about 10² to 10³-fold higher than that obtained from HSV-1 (F)-infected J-uPAR cells.

(iii) The concentration of FBS did not affect the replication of R5181 virus in J-uPAR cells.

(iv) The titer obtained from infected J-uPAR cells infected with R5181 mutant virus was 10- to 100-fold lower than that obtained from J-uPAR cells infected with R5182 mutant virus.

(v) Unexpectedly, the titer obtained from ATF-uPA-gD virus-infected J1.1 cells was of the same order as that obtained from ATF-uPA-gD virus-infected J-uPAR cells.

II. Isolation and Reverse Transcription of RNA Extracted from J1.1 and J-uPAR Cells.

The experiments described above indicated that R5181 virus replicated in J1.1 cells. One possible explanation for these results is that J1-1 cells express an endogenous hamster uPAR. Consistent with this hypothesis is the evidence reported elsewhere that human uPA binds to hamster uPAR with high affinity (Fowler, B., et al., 1998). To test this hypothesis, two series of experiments were performed. In the first, J1.1 cells were tested to determine if they express hamster uPAR mRNA.

Total RNA was extracted with the aid of TRIzol reagent according to the manufacturer's instructions (Life Technologies, Rockville, Md.). DNase I treatment (Life Technologies), phenol/chloroform extraction, and ethanol precipitation (Fisher Scientific, Houston, Tex.) were carried out to remove possible DNA contamination. Total RNA (3 μl) was reverse-transcribed to yield single-stranded cDNA with 20 U of AMV reverse transcriptase (Promega) in a total reaction volume of 30 μl. The reverse transcription was primed with the following primers:

| Human uPAR reverse: | |
|---|---|
| 5′-GGCAGTCATTAGCAGGGTGATGGTG- | (SEQ ID NO: 57) |
| 3′ | |
| Hamster uPAR reverse | |
| 5′-GTTGCCCTCGCAGCTGTAACACTGG- | (SEQ ID NO: 58) |
| 3′ | |

Reverse transcription was performed using a pool of nucleotides consisting of 10 mM concentrations (each) of dGTP, dATP, dTTP, and dCTP (Promega). Forty units of RNasin (Promega) were added to each reaction mixture. The mixture containing only the RNA template, and the primer was first heated at 70° C. for 10 minutes, chilled on ice, and after the addition of the other components, incubated at 42° C. for 30 minutes, shifted to 52° C. for 30 minutes, and then heat-inactivated at 95° C. for 5 minutes. cDNAs obtained from reverse transcription of RNA extracted from J1.1 and J-uPAR cells were amplified by PCR under the following conditions: 1 minute at 94° C., 1 minute at 60° C., and 75 seconds at 72° C. The following primers were used for PCR:

| | |
|---|---|
| uPAR-start forward: | |
| 5′-ATGGGTCACCCGCCGCTGCTGCCGC-3′ | (SEQ ID NO: 59) |

Human uPAR reverse primer or hamster uPAR reverse primer used in PCR were as described above. As shown in FIG. 6, the J1.1 cell line contained detectable levels of hamster uPAR mRNA but not human uPAR mRNA.

In the second series of experiments, human single-chain uPA was used to compete with the virus for the putative hamster uPAR receptor on J1.1 cells. Human single chain uPA (scuPA) was purchased from American Diagnostics Inc. (Stamford, Conn.). Cells were exposed to increasing concentrations of the human scuPA for 60 minutes at 37° C. and then exposed to 0.1 pfu of R5181 for 120 minutes at 37° C. in the presence of human scuPA. After 24 hours of incubation, the cells were harvested, sonicated and titrated on Vero cells.

Previous studies showed that human scuPA bound to human uPAR with high affinity (Kd of about 1 nM; Roldan, A. L., et al., 1990; Barnathan, E. S., et al., 1990) and human scuPA binds to hamster uPAR with the same affinity as for the human receptor (Kd=11.1 nM) (Fowler, et al., 1998). The J1.1 cells exposed to the highest concentration of scuPA produced 10-fold less virus than control untreated cells (FIG. 7). The results (FIG. 7) showed that scuPA competed with the virus more successfully in J1-1 cells than in J-uPAR cells, consistent with the expectation that J-uPAR cells would have more receptors than J1.1 cells.

III. Treatment with Endosome Inhibitors.

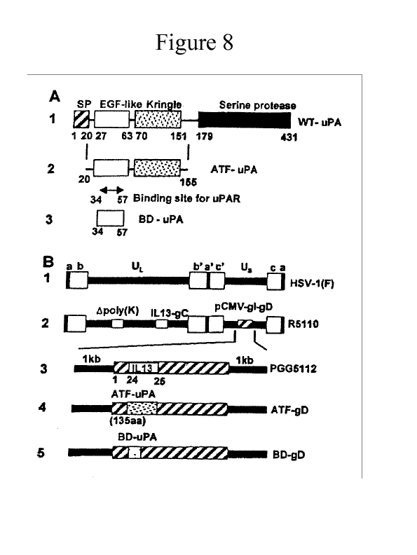

Cells were exposed to freshly prepared NH₄Cl at the concentrations indicated in FIG. 8 for 30 minutes at 37° C. and then exposed to 0.01 pfu of R5181 virus or HSV-1(F) per cell in the presence of the NH₄Cl. After 24 hours of incubation, the cells were harvested, sonicated and titrated on Vero cells. The entry of R5181 virus into J-uPAR or J1.1 cells was not inhibited by NH₄Cl even at the highest concentration tested (FIG. 8). The results indicated that the R5181 virus does not depend on the endocytic pathway as a primary mechanism for entry into J-uPAR cells.

The preceding Examples describe the construction and properties of recombinant viruses designed to target cells expressing uPAR, with the results summarized below.

(i) R5181 virus infected and replicated in cells exhibiting uPAR. R5182 virus was unable to infect cells via uPAR. It is unknown whether the R5182 virus failed to infect cells via uPAR because the secondary structure of the chimeric gD blocked the binding site from interacting with uPAR or whether the insert was incompatible with the predicted modification of gD following its interaction with a receptor.

(ii) Two lines of evidence indicated that R5181 entered J1.1 cells by interacting with uPAR. Foremost, scuPA competed with R5181 virus for entry into J1.1 cells. The second line of evidence is based on studies that indicated that virions may undergo endocytosis in the absence of a cell-surface receptor. The results herein (e.g., FIG. 8) show that endocytosis was not the major mechanism whereby R5181 entered J1.1 or J-uPAR cells inasmuch as it was unaffected by NH₄Cl. In contrast, NH₄Cl reduced the already low yield of HSV-1 (F) by approximately 10-fold in both J1.1 and J-uPAR cells which is consistent with the expectation that a small amount of virus may enter cells by endocytosis in the absence of a specific receptor.

(iii) Cells transduced with uPAR required special care in the maintenance of the receptor on serial passage. The stability of the receptor was dependent on cell type, serum concentration, and, as in the studies described herein, the method by which the cells were dislodged for serial passage. Although this study began with the premise that BHK TK⁻ cells would not exhibit an uPA receptor, BHK TK⁻ cells that were passaged numerous times by a variety of means still expressed the endogenous receptor, albeit at levels significantly lower than that that of transduced cells. Thus, receptors may not always be present in tumor cells passaged serially in culture or they may be unstable in transduced cells.

(iv) Ligands or other binding pair members may be inserted into the HSV-1 virion to provide a mode of entry into cells. Additionally, binding pair members may be targeted to receptors that are not anchored via their own transmembrane domain.

### EXAMPLE 10

Supplemental Materials and Methods

The experimental work described in Examples 11-16, below, were conducted in general conformity with the materials and methods described above. In addition, the following materials and methods were also used in conducting these experiments.

Cells. Vero and HEK293 were obtained from the American Type Culture Collection (Manassas, Va.). R6 cells were derived by transduction of rabbit skin cells with plasmid pEA102 containing the HSV-1 gD coding sequence under the U_{L}26.5 promoter (Zhou 2000). R6 cells expresses gD and enable ΔgD viruses to replicate and spread from cell to cell by complementing the virions with gD made ectopically. The thymidine kinase minus J1.1 cell line lacking all receptors for wild-type virus, J-HveA and J-nectin cell lines the kind gifts of G. Campadelli-Fiume, Univ. of Bologna, Italy (Zhou 2002). The properties of the Vero13R cell line expressing the IL13α2 receptor were described elsewhere (Zhou 2005).

Viruses. HSV-1(F) is the prototype wild-type virus used in this laboratory. The recombinant viruses described in this report were generated by co-transfection of R6 cells with a plasmid containing the desired construct of chimeric glycoprotein D and intact DNA of the ΔgD virus R5110 as described elsewhere (Zhou 2002). The progeny of transfection was then plated on R6 cells, plaque purified and the progeny virus. The gD contained in the virion was then analyzed to insure that it contained the sequence of the desired gD construct. The plasmids encoding gD constructs and used for production of recombinant viruses are described in result and were made by standard procedures described elsewhere (Zhou 2002).

Virus replication in cell lines. Replicate cultures of J-HveA, J-Nectin or Vero13R cells were exposed to 0.1 PFU of recombinant viruses or HSV-1 (F) per cell. After 24 hours at 36° C., the cells were harvested, disrupted by sonication. Viral progeny was titered on Vero13R cells.

Transfection plasmids. Transfer vector pAc-CMV, which contains the CMV-IE promoter-enhancer sequences in the XhoI-BamHI sites of pAc-SG2, was described elsewhere (Zhou and Galvan 2000). To construct pGG5414, pGG5415, pGG5416, an EcoRI-PstI fragment was derived by PCR from R5321, R5322 and R5323 respectively with primers 5′-CGGAATTCATGGGGGGGGC TGCCGCCAG-3′ (for 5414; SEQ ID NO:60), 5′-CGGAATTCATGGCGCTTTTG T TGACCACGG-3′ (for 5415; SEQ ID NO:61), 5′-CGGAATT CATGAGAGCC CTGCTGGCGCGCC-3′ (for 5416; SEQ ID NO:62), along with 5′-AACTGCAGCTAG GCGTAGTAAACCGTGATCGGG-3′ (SEQ ID NO:63) and then inserted into the EcoRI-PstI sites of pAc-CMV transfer vector. To construct the transfer vector expressing residues 219-314 of mature gD with Myc tag, an EcoRI-PstI fragment was amplified by PCR from gD-A (Zhou and Roizman 2001) with primers 5′-CGGAATTCATGCTGCCCCGCTTCATCCCCGAG-3′ (SEQ ID NO:64) and 5′-AACTGCAGTTACAGGTC CTCCTCTGAGATCAGCTTCTGCATTGATGCGTTGTTCGGGGTGGCCGGGGG-3′ (SEQ ID NO:65) and then inserted into the EcoRI-PstI sites of pAc-CMV. Transfer vector pGG5418 was constructed the uPA ATF fragment into the EcoRI-PstI sites of pAc-CMV transfer vector by PCR amplification using the primers 5′-CGGAATT CATGAGAGCC CTGCTGGCGCGCC-3′ (SEQ ID NO:66) along with 5′-AACTGCAGCTATTTTCCATCTGCGCAGTCATGC-3′ (SEQ ID NO:67).

Antibodies. The ZC25 antibody to the C-terminal domain of gD was the kind gift of G. H. Cohen and R. J. Eisenberg (Zhou 2006). Monoclonal antibodies against gD (clone H170) and ICP0 (clone H1083) were from the Goodwin Institute, Plantation, Fla. Monoclonal antibodies against IL13 protein and described previously (Zhou 2002). Anti Myc antibody were from and Santa Cruz Biotechnology. Monoclonal antibodies against human ATF of uPA (Kamiyama 2006) were purchased from American Diagnostica Inc. (Stamford, Conn.).

Communoprecipitation assays. Subconfluent cultures of HEK293 cells in 25-cm² flasks were co-transfected with 1 μg of pGG5417 ands 1 μg of pGG5414, pGG5415 or pGG5416. The cells were harvested 40 hours after transfection, collected by centrifugation, rinsed twice with 5 ml of PBS, resuspended in 200 μl of lysis buffer (20 mM Tris [pH 8.0], 1 mM EDTA, 1% NP-40, 400 mM NaCl, 2 mM dithiothreitol [DTT], 0.1 mM NaVO₄, 10 mM NaF, 1× protease inhibitor cocktail [Sigma, St. Luis, Mo.]), and chilled on ice for 40 minutes. The supernatant fluid (150 μl) collected after centrifugation at 1,000 rpm for 2 min (Eppendorf, 5415C) was diluted with 150 μl of low-salt lysis buffer (20 mM Tris [pH 8.0], 1 mM EDTA, 1% NP-40, 16 mM NaCl, 2 mM DTT) and incubated with 5% rabbit preimmune serum at 4° C. for 1 hour, followed by incubation with 50 μl of protein A-Sepharose for 1 hour at 4° C. and centrifugation at 3,000 rpm for 3 minutes to remove nonspecifically bound proteins. The supernatants fluids were reacted with ant-Myc monoclonal antibody at 4° C. for 16 hours, and then reacted with 20 μl of protein A-Sepharose at 4° C. for 1 hour. Immune complexes bound to protein A-Sepharose were rinsed three times with rinse buffer (50 mM Tris [pH 7.4], 10 mM MgCl₂, 5 mM DTT), collected by centrifugation disrupted by boiling in sample buffer for 5 minutes, subjected to electrophoresis on denaturing gels, transferred to a nitrocellulose sheet and probed with monoclonal antibodies to gD(H170), IL13 or uPA. In parallel, aliquots of the supernatant fluids were reacted with antibodies to gD(H170), IL13 or uPA. The collected immune precipitates were subjected to electrophoresis in denaturing gels, transferred to a nitrocellulose sheet and probed with antibody to the Myc epitope.

### EXAMPLE 11

Construction and Infection

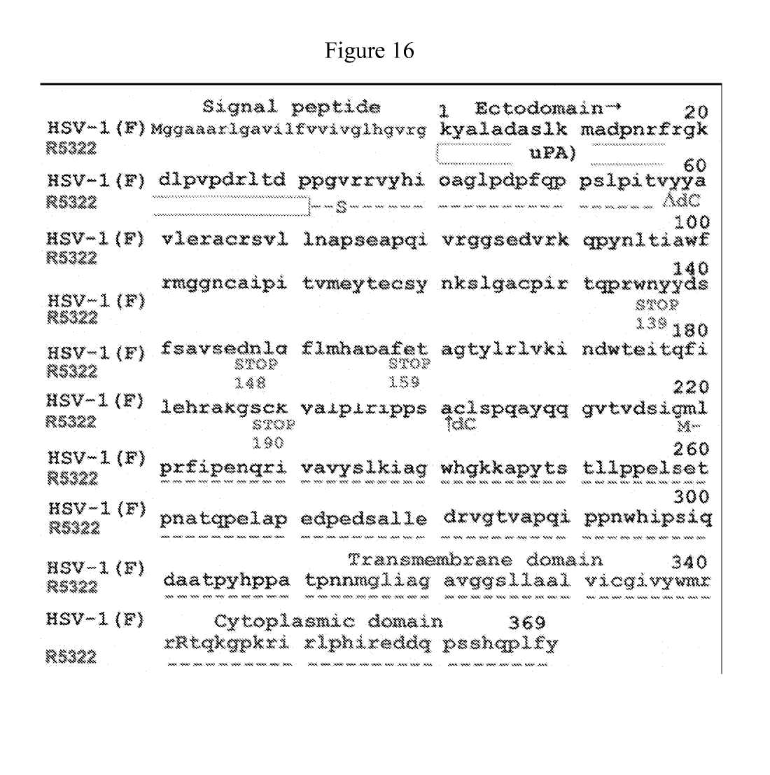

Studies conducted with the HSV constructs described herein have shown that residues 61 through 218 (all numbering herein is the numbering for mature gD; for the nascent gD sequence of SEQ ID NO:26, add 25) of the wild-type gD glycopolypeptide are dispensable for viral entry. The design of the R5322 recombinant virus involved replacement of residues 1-32 with the N-terminal 155 residues of uPA and substitution of valine 34 with serine but retention of the signal sequence of wild-type gD. R5322 was generated by co-transfecting R6 cells with intact R5110 DNA which lacks the gD gene and a plasmid encoding the desired chimeric gD. R6 cells were derived by transduction of rabbit skin cells with plasmid pEA102 containing the HSV-1 gD coding sequence under the U_{L}26.5 promoter. These R6 cells express gD ectopically and enable viruses lacking gD to replicate and spread (15). The infectious virus was plaque purified and initial stocks were produced in R6 cells. To verify the presence of the N-terminal domain of uPA, the recombinant gD was amplified by PCR and sequenced. Repeated sequencing concurrently with appropriate wild-type controls revealed that in gD of recombinant R5322 one cytosine was deleted after codon 60 and one was inserted after codon 201 (FIG. 16). The frameshifts resulting from the deletion and insertion of the cytosines introduced stop codons at residues 139, 148, 159, and 190. The first potential initiator methionine is at position 219 of mature gD. Further tests showed that the virus infected and replicated in 4 test cell lines, i.e., in J1.1, a hamster cell line that lacks receptors for wild-type virus; J-HveA, a hamster cell line that expresses the HveA receptor; J-Nectin, a hamster cell line that expresses nectin 1; and Vero13R, a Vero derived cell line that expresses IL13α2 receptor in addition to the natural receptors for HSV-1. The observation that R5322 replicated in J1.1 cells is consistent with earlier results showing that these cells accumulate uPAR mRNA (19).

### EXAMPLE 12

Fusion Variants

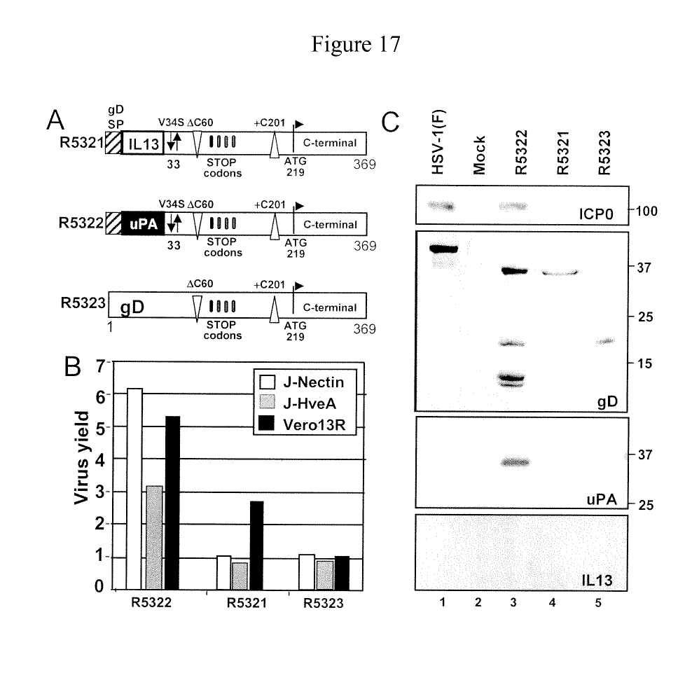

The results disclosed in Example 10 raised the question whether residues 60-218 of gD are dispensable for entry just of this virus or whether they play no essential role in wild-type virus as well. To answer this question, two additional viruses were constructed and designated R5321 and R5323. To produce R5321, R6 cells were transfected with R5110 DNA and a chimeric gD construct that was identical to that of R5322 except that the N-terminal domain of uPA was replaced with that of IL13 (19). In the chimeric plasmid designed to generate R5323 the residues uPA-gD₃₃₋₆₀ of R5322 were replaced with residues 1-60 of wild-type gD. Schematic representations of the chimeric gDs are shown in FIG. 17, Panel A. Viral stocks were made and characterized as described herein and tested by exposing replicate 25 cm² cultures of J-HveA, J-Nectin, or Vero13R cells to 0.1 PFU/cell. The progeny viruses were titered in Vero13R cells. gD ectopically expressed in R6 cells enabled the amplification of recombinants produced in these cells. The spread and replication of recombinant viruses in J-HveA, J-Nectin or Vero13R cells were dependent on the presence of an appropriate receptor binding domain in the chimeric gD. As shown in FIG. 17, Panel B, R5322 replicated in all cell lines to approximately the same extent as the R5181 recombinant virus in which the chimeric gD consisted of the uPA sequences inserted after codon 24 of gD (19). The recombinant R5321 replicated to a very low level and only in Vero13R cells (FIG. 17, panel B). The products of transfection used to generate R5323 did not recombine to form an infectious virus.

### EXAMPLE 13

Expression

To determine whether the recombinant viruses expressed the appropriate protein, Vero13R cells were exposed to 1.0 PFU of wild-type HSV-1 (F), R5321, R5322 or R5323 viruses. As shown in FIG. 17, Panel C, the only protein detected in lysates of R5323-infected cells was a truncated form of gD. Cells infected with either wild-type virus or the R5322 mutant produced ICP0, a major α (immediate early) regulatory protein of the virus. As could be expected from the presence of in-frame stop codons, the major product of R5322 virus reacting with either gD or uPA antibody was significantly smaller than the wild-type gD made in wild-type virus-infected cells, consistent with the hypothesis that the N-terminal and C-terminal domains of gD were produced independently.

### EXAMPLE 14

Constructs with Internal P_{CMV}

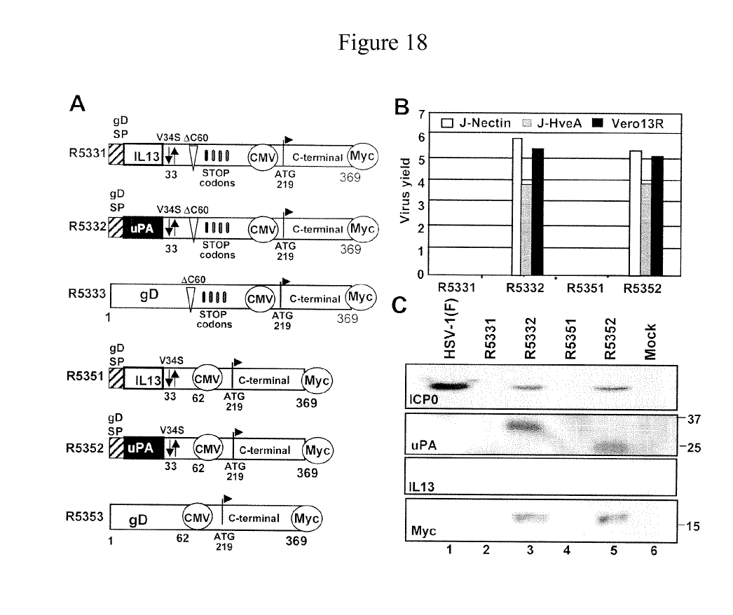

HSV-1 infected cells can suppress one stop codon at a very low level (21). To make a single protein based on the sequence of the chimeric gD in R5322, all 4 stop codons would have to be suppressed. An alternative explanation is that a promoter domain within the gD ORF enables the synthesis of mRNA that encodes a functional protein starting with methionine 219 (mature gD). Implicit in this hypothesis is the notion that residues 61 to 218 simply link the N-terminus of gD to the C-terminal domain but that the sequence itself is important only in the sense that it maintains the two ends in the proper relationship. To investigate these observations, two sets of viruses were constructed (FIG. 18).

The viruses predicted to be formed by recombinant gD constructs designated R5331, R5332 and R5333 differed from the corresponding recombinant gD constructs in R5321, R5322, and R5323, in that a human cytomegalovirus immediate early (CMV-IE) promoter was inserted upstream of residue 219 (mature gD). In recombinant gD constructs designed to make R5351, R5352 and R5353, residues 63-218 were deleted, and a stop codon and the CMV-IE promoter were inserted between residues 62 and 219 (mature gD). In addition, a Myc epitope tag was inserted at the C-terminus of gD to enable identification of the protein made from the corresponding domain of chimeric gD.

Recombinant viruses R5332 and R5352 that were produced and titered in R6 cells were used to infect other cell lines at a ratio of 0.1 PFU/cell. As shown in FIG. 18, Panel B, these recombinant viruses replicated in J-Nectin, J-HveA and Vero13R cell lines to the same extent as R5322 (see Panel B of FIG. 17). R6 cells transfected with chimeric gD constructs designed to produce the recombinant viruses R5331 or R5351 exhibited extensive cytopathic effects. However, the lysates of these cells did not replicate in the 3 test lines (J-HveA, J-Nectin, or Vero 13R). No infectious virus progeny of the transfection of R6 cells with R5333 or R5353 were detected and these constructs were not tested further.

### EXAMPLE 15

Western Analyses

Lastly, analyses of key proteins made by the recombinant viruses constructed in this series of experiments are shown in FIG. 18, Panel C. Vero13R cells infected with either R5332 or R3552 accumulated two sets of co-migrating bands, one that reacted with antibody to ICP0 and one that reacted with anti-Myc antibody. The anti-uPA antibody reacted with a protein band in R5332-mutant virus-infected cells that migrated more slowly than the protein detected by the anti-uPA antibody in R5352 mutant virus-infected cells. We conclude that (i) the tagged gD proteins containing the residues 219 to 369 (mature gD) were expressed as separate entities from the proteins reactive with the anti-uPA antibody and (b) the differences in electrophoretic mobility of proteins reactive with the anti-uPA antibody reflect their sizes. Thus, in R5352 mutant virus-infected cells, the N-terminal polypeptide comprises uPA and gD residues 33 to 60. In R5332 mutant virus-infected cells, translation of the mRNA proceeds beyond codon 60 to at least the first stop codon at position 139.

We conclude that (i) the residues 61 to 218 of gD are not required for entry into cells, (ii) the essential N-terminal and C-terminal domains of gD can be produced independently of each other, and (iii) infectivity depends on the nature of the ligand attached to the N-terminal domain of gD.

### EXAMPLE 16

Peptide Interactions

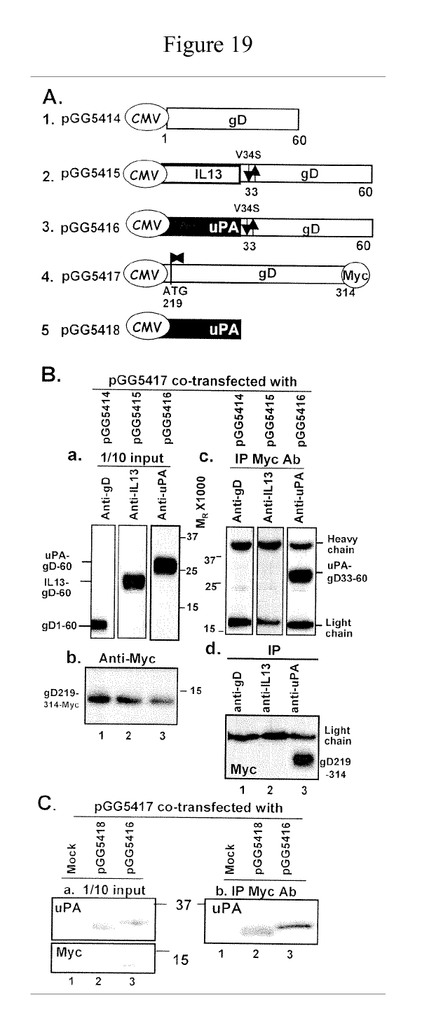

The N-terminal domain of gD linked to uPA co-precipitated with the C-terminal domain of gD. The results shown in FIG. 18 indicated that in R5332 or R5352 infected cells the amino acid stretch comprising the N-terminal domain of uPA linked to residues 33-60 of gD were in a different polypeptide (uPA-gD₃₃₋₆₀) than the C-terminal domain of gD consisting of residues 219 to 369 (gD₂₁₉₋₃₆₉, numbering of mature gD). To infect cells, however, the two functional domains of the chimeric gD had to interact. To determine whether the two polypeptides interact, we constructed 4 plasmids schematically illustrated in FIG. 19, Panel A. Plasmid pGG5414 encoded residues 1-60 of gD (SEQ ID NO:26) driven by the CMV promoter. Plasmids pGG5415 and pGG5416 consisted of uPA-gD₃₃₋₆₀ and IL13-gD₃₃₋₆₀ driven by the CMV promoter, respectively. In each of the latter two plasmids, the valine codon 34 was substituted with that of serine. Finally, plasmid R5417 contained mature gD residues 219-314, i.e., gD without the transmembrane and cytoplasmic domains tagged at the C-terminus with the Myc epitope and driven by the CMV promoter. The plasmids were transfected into HEK293 cells in pair-wise fashion, i.e., pGG5417 with either pGG5414, pGG5415 or pGG5416. The cells were harvested 40 hours after transfection, lysed and reacted with antibodies to either gD (transfection of pGG5417+pGG5414), IL13 (pGG5417+pGG5415), uPA (pGG5417+pGG5416) or Myc. As shown in Panel Bc of FIG. 19, anti-Myc antibody precipitated a protein reactive with the anti-uPA antibody. Conversely, as shown in panel Bd, the anti-uPA antibody precipitated a protein reactive with Myc. Anti-Myc antibody did not pull down proteins reactive with the mature gD₂₁₉₋₃₁₄ polypeptide from cells transfected with pGG5417+pGG5414, or pGG5417+pGG5415. The results are described using the numbering of mature gD and show that the uPA-gD₃₃₋₆₀ polypeptide physically interacts with the gD₂₁₉₋₃₁₄ polypeptide. No evidence has been found that either IL13-gD₃₃₋₆₀ or wild-type gD1-60 interact physically with the gD₂₁₉₋₃₆₀ polypeptide. One explanation for the failure to detect infectious R5333 or R5353 virus is the failure of the two key components of gD to interact with each other.

One explanation of our results is that the co-precipitation of uPA-gD₃₃₋₆₀ (mature gD numbering) with gD₂₁₉₋₃₆₉ (mature gD numbering) reflects the interaction of a Kringle domain (residues 50 to 132) in the N-terminal domain of uPA with lysines in the C-terminal domain of gD. To test this explanation, HEK293 cells were transfected with mixtures of pGG5417 and a plasmid (pGG5418) encoding the N-terminal domain of uPA or pGG5417 and pGG5416. The lysates of the transfected cells were reacted with antibody to Myc. The immune precipitates were collected, electrophoretically separated in a denaturing gel, transferred to a nitrocellulose membrane and reacted with anti-uPA antibody. As shown in FIG. 19, panel Cb, anti-Myc antibody co-precipitated uPA from lysates of cells co-transfected with pGG5417 and pGG5418 (lane 2) and uPA 119-314 from lysates of cells co-transfected with pGG5417 and pGG5416 (lane 3).

An infectious virus with two frameshifts, one after codon 60 and the second after codon 201, was constructed. The frameshifts replaced the codons 61 through 201 with a sequence that contained 4 stop codons. The salient feature of the recombinant virus that emerged from these studies is that a virus with a gD consisting of polypeptide A containing the N-terminus of uPA fused to residues 33-60 of gD and a polypeptide B containing the C-terminal domain of gD was infectious. Attempts to construct recombinant viruses in which the A polypeptides consisted solely of gD residues 1-60 or of the 132 residues encoding IL13 fused to gD residues 33 to 60 were not infectious. These results led to the following conclusions, stated using the numbering of mature gD:

(i) Residues 61 to 218 of gD do not execute a function required for HSV-1 entry into cells. They appear to serve as a linker between the N-terminal targeting domain and the C-terminal pro-fusion domains of gD. It is of interest to note that residues 61-218 coincide almost entirely with the immunoglobulin-like core of gD located between residues 56 and 184 (7). In light of these results, the primary function of the gD core appears to be to hold the key N-terminal and C-terminal domains in proper orientation. This domain may also be required to block cell death as a consequence of a discharge of lysosomal enzymes (17).

(ii). The fundamental difference between the constructs which yielded an infectious virus and those that failed is that polypeptide A, consisting of uPA₃₃₋₆₀, interacted with the C-terminal domain of gD, whereas those containing gD1-60 or IL13-gD₃₃₋₆₀ did not. The evidence supports the conclusion that uPA itself can interact with gD₂₁₉₋₃₁₄. The results support the conclusion that physical interaction of the domain capable of binding a cell surface receptor with the C-terminal domain of gD may lead to successful virus entry into cells whereas lack of physical interaction may lead to failure. The results reinforce the conclusion that entry requires the physical rapprochement of gD to the cell surface receptor.

(iii) Current studies indicate that interaction of gD with its receptors alters its conformation and exposes the C-terminus of gD to fusogenic glycoproteins gB, gH and gL (23, 24, 25). It is unlikely that each of the three ligands selected by chance alters the conformation of the remaining portions of gD in an identical manner to enable virus entry. One hypothesis that remains to be explored is that gD is “armed” by a conformational change resulting from replacement of residues 1-32 by any of the 3 ligands, and that cell membrane-envelope fusion is executed when the ligands interact with their cognate receptors. A corollary of this hypothesis is that while the interaction of the receptor binding domain with its natural cell surface receptor may alter the conformation of the distal portions of gD, such changes are not obligatory consequences of the interaction of chimeric gD molecules with novel cognate receptors.

Apparent from the experimental results disclosed above, the invention comprehends a herpes simplex virus providing an expressible coding region in which a coding sequence for a targeting peptide of interest is translationally fused to a peptide fusion containing a sequence encoding mature gD 33-60 fused to a peptide interaction domain capable of specific interaction with mature gD 219-369. An exemplary peptide interaction domain is a Kringle domain, such as the Kringle domain of uPA residues 50-132, shown to interact with the C-terminal fusogenic domain of gD. In fusing a coding region for a targeting peptide of interest to a coding sequence for the peptide fusion, the targeting peptide is freed from a role in the interaction of gD domains to effect HSV entry. Thus, the only role for such a targeting peptide is to specifically recognize its binding partner, thereby providing maximal flexibility in the design of a viral vector amenable to customized targeting to a cell of interest.

Expanding upon the aspect of the invention described immediately above, the uPA ligand described above might be replaced by any of a number of alternative ligands in trans, provided that they can associate with the pro-fusogenic domain, to extend the host range of recombinant viruses in useful ways. More generally, the invention comprehends an engineered interaction between the fused targeting peptide-gD interaction domain and the gD fusogenic domain. Based on the disclosure herein that the N-terminally disposed gD interaction domain interacts with the C-terminally disposed fusogenic domain in effecting HSV entry, constructs according to the invention can rely on naturally occurring domains or motifs, such as the Kringle domain and the polylysine region, to ensure interaction of these two gD domains or reliance can be placed on any interacting domains/motifs that are engineered as part of fusion polypeptides. One fusion polypeptide comprises a targeting peptide, the N-terminally disposed gD interaction domain (e.g., mature gD residues 33-60), and an interacting domain/motif, the other polypeptide comprises a fusogenic domain of mature gD (e.g., residues 219-369) and the cognate member of an interacting pair of domains/motifs. The two polypeptides may be part of a single protein chain or they may be separate chains. As with the other inventive constructs described herein, linkers may be interposed between any and all elements of the polypeptides to provide spacing and flexibility, using routine procedures known in the art.

From the foregoing it will be appreciated that, although specific embodiments of the invention have been described herein for purposes of illustration, various modifications may be made without deviating from the spirit and scope of the invention.

### REFERENCES

Davis F G, Freels S, Grutsch J, Barlas S, Brem S. (1998) J. Neurosurg. 88:1-10.
Pyles R B, Warnick R E, Chalk C L, Szanti B E, Parysek L M. (1997) Hum Gene Ther. 8(5):533-44.
Rampling R, Cruickshank G, Papanastassiou V, Nicoll J, Hadley D, Brennan D, Petty R, MacLean A, Harland J, McKie E, Mabbs R, Brown M. (2000) Gene Ther. 7(10):859-66.
McKie E A, Brown S M, MacLean A R, Graham D I. (1998) Neuropathol Appl Neurobiol. 24(5):367-72.
Markert J M, Medlock M D, Rabkin S D, Gillespie G Y, Todo T, Hunter W D, Palmer C A, Feigenbaum F, Tornatore C, Tufaro F, Martuza R L. (2000) Gene Ther. 7(10):867-74.
Mineta T, Rabkin S D, Yazaki T, Hunter W D, Martuza R L. (1995) Nat Med. 1(9):938-43.
Simard C, Langlois I, Styger D, Vogt B, Vlcek C, Chalifour A, Trudel M, Schwyer M. (1995) Virology. 212(2):734-40.
Chou J, Chen J J, Gross M, Roizman B. (1995) Proc Natl Acad Sci USA. 92(23):10516-20.
He B, Chou J, Brandimarti R, Mohr I, Gluzman Y, Roizman B. (1997) J Virol. 71(8):6049-54
Cassady K A, Gross M, Roizman B. (1998) J Virol. 72(9):7005-11.
Leib, D. A., Harrison, T. E., Laslo, K. M., Machalek, M. A., Moorman N. J. and Virgin, H. A W. (1999) J. Exp. Med. 189:663-672.
Laquerre S, Argnani R, Anderson D B, Zucchini S, Manservigi R, Glorioso J C. (1998). J Virol. 72(7):6119-30.
Spear, P. G., R. J. Eisenberg, and G. H. Cohen. (2000) Virology 275: 1-9.
Montgomery, R. I., M. S. Warner, B. J. Lum, and P. G. Spear. (1996) Cell 87:427-436.
Campadelli-Fiume, G., F. Cocchi, L. Menotti, and M. Lopez. (2000) Reviews in Medical Virology. 10:305-319.
Zhou G, Roizman B. (2002) J Virol. 76(12):6197-204.
Debinski W, Gibo D M, Hulet S W, Connor J R, Gillespie G Y. (1999) Cancer Res. 5:985-990.
Mintz A, Gibo D M, Slagle-Webb B, Christensen N D, Debinski W. (2002) Neoplasia 4:388-399.
Debinski W. (1998) Crit. Rev. Oncogen. 9:255-268.
Debinski W, Gibo D M. (2000) Mol. Med. 6:440-449.
Zhou G, Roizman B. (2001) J Virol. 75(13):6166-72.
Arsenakis M, Tomasi L F, Speziali V, Roizman B, Campadelli-Fiume G. (1986) J Virol. 58(2):367-76.
Ye G J, Roizman B. (2000) Proc Natl Acad Sci USA. 97(20):11002-7.
Zhou G, Galvan V, Campadelli-Fiume G, Roizman B. (2000) J Virol. 74(24):11782-91.
Carfi A, Willis S H, Whitbeck J C, Kirummenacher C, Cohen G H, Eisenberg R J, Wiley D C. (2001) Mol Cell. 8(1):169-79.
Cocchi, F., Menotti, L., Mirandola, P and Campadelli-0Fiume, G. (1998) J. Virol. 72:9992-10002.
Debinski W, Thompson J P. 1999. Clin Cancer Res. 5(10 Suppl):3143s-3147s.
Brooks, P. C., Clark, R. A. F., and Cheresh, D. A. Requirement of vascular integrin αᵥβ₃ for angiogenesis. Science 264:569-571, 1994.
Brooks, P. C., Montgomery, A. M. P., Rosenfeld, M., Reisfeld, R. A., HU, T., Klier, G., and Cheresh, D. A. Integrin αᵥβ₃ antagonists promote tumor regression by inducing apoptosis of angiogenic blood vessels. Cell 79:1157-1164, 1994.
Burger, M. J., Tebay, M. A., Keith, P. A., Samaratunga, H. M., Clements, J., Lavin, M. F., and Gardiner, R. A. Expression analysis of δ-Catenin and prostate-specific membrane antigen: Their potential as diagnostic markers for prostate cancer. Int. J. Cancer 100:228-237, 2002.
Ellerman, T. C., Domagala, T., McKern N. T. et al, Identification of the determinant of Epidermal growth factor receptor ligand-binding specificity using truncated, high affinity form of the ectodomain. 2001 Biochemistry 40 8930-8939.
Genbitsky, D. S., Bozso, Z., O'Flaharty, M. et al., 2001 A specific binding site for a fragment of the B-loop of epidermal growth factor and related peptides. Peptides 23:97-102 A.
Urbanelli, L., Ronchini, C., Fontana, L. et al., Tergeted gene transduction of mammalian cells expressing the HER2/neu receptor by filamentous phage. J Mol Biol. 2001 Nov. 9; 313(5):965-76.
Hayashi, T., Takahashi, T., Motoya, S., et al. MUC1 Mucin core protein binds to the dfomasin of ICAM-1 2001 Digestion 63:87-92.
Fracasso, G., Bellisola, G., Cingarlini, S., Castelletti, D., Prayer-Galletti, T., Pagano, F., Tridente, G., and Colombatti, M. Anti-tumor effects of toxins targeted to the prostate specific membrane antigen. Prostate 53:9-23, 2002.
Mabjeesh, N. J., Zhong, H., and Simons, J. W. Gene therapy of prostate cancer: current and future directions. Endo. Related Cancer 9:115-139, 2002.
Ross, S., Spencer, S. D., Holcomb, I., Tan, C., Hongo, J., Devaux, B., Rangell, L., Keller, G. A., Schow, P., Steeves, R. M., Lutz, R. J., Frantz, G., Hillan, K., Peale, F., Tobin, P., Eberhard, D., Rubin, M. A., Lasky, L. A., and Koeppen, H. Prostate stem cell antigen as therapy target: Tissue expression and in Vivo efficacy of an immunoconjugate. Cancer Res. 62:2546-2553, 2002.
Ruoslahti, E. RGD and other recognition sequences for integrins. Annu. Rev. Cell Dev. Biol. 12:697-715, 1996.
Thomas, J., Gupta, M., Grasso, Y., Reddy, C. A., Heston, W. D., Zippe, C., Dreicer, R., Kupelian, P. A., Brainard, J., Levin, H. S., and Klein, E. A. Preoperative combined nested reverse transcriptase polymerase chain reaction for prostate-specific antigen and prostate-specific membrane antigen does not correlate with pathologic stage or biochemical failure in patients with localized prostate cancer undergoing radical prostatectomy. J. Clin. Oncol. 20:3213-3218, 2002.
Lorimer and Lavictoire, Targeting retrovirus to cancer cells expressing a mutant EGF receptor by insertion of a single chain antibody variable domain in the envelope glycoprotein receptor binding lobe, J Immunol Methods 237(1-2):147-57, 2000.
Turner et al., Glycoproteins gB, gD, and gHgL of Herpes Simplex Virus Type 1 are Necessary and Sufficient t Mediate membrane fusion in a Cos cell transfection system, J of Virol, 72(1): 873-75, 1998.
Brunetti et al., Herpes Simplex Virus gD and Virions Accumulate in Endosomes by Mannose 6-Phosphate-Dependent and -Independent Mechanisms, J of Virol, 72(4):3330-3339, 1998.
Sharma et al., Molecular imaging of gene expression and protein function in vivo with PET and SPECT, J Magn Reson Imaging, 16(4):336-51, 2002.
Vries et al., Scintgraphic Imaging of HSVtk Gene Therapy, Curr Pharm Des, 8(16):1435-50, 2002.
Vries et al., Positron emission tomography: measurement of transgene expression, Methods, 27(3):234, 2002.
Soling et al., Intracellular localization of Herpes simplex virus type 1 thymidine kinase fused to different fluorescent proteins depends on choice of fluorescent tag, FEBS Lett, 527(1-3):153, 2002.
Zhou G., and Roizman B., J Virol, 9:5272-5277 (2005)
Manoj S., et al., Proc. Natl. Acad. Sci. USA., 101: 12414-12421 (2004);
Zago A., et al., Proc. Natl. Acad. Sci. USA., 101:17498-17503 (2004);
Connolly S A., J. Virol., 79:1282-1295 (2005)
2. Spear, P. G. (2004) Cell Microbiol 6, 401-410.
3. Spear, P. G. & Longnecker, R. (2003) J. Virol 77, 10179-10185.
6. Fusco, D., Forghieri, C. & Campadelli-Fiume, G. (2005) Proc. Natl. Acad. Sci. USA 102, 9323-9328.
7. Kirummenacher, C., Supekar, V. M., Whitneck, J. C., Connolly, S. A., Eisenberg, R. J., Cohen, G., Wiley, D. C. & Carfi, A. (2005) EMBO J 24, 4144-4153.
8. Cocchi F, Fusco D, Menotti L, Gianni T, Eisenberg R J, Cohen G H, Campadelli-Fiume G. (2004). Proc. Natl. Acad. Sci. USA 101, 7445-7450.
9. Willis S H, Rux A H, Peng C, Whitbeck J C, Nicola A V, Lou H, Hou W, Salvador L, Eisenberg R J, Cohen G H. (1998). J. Virol. 72, 5937-47.
10. Connolly S A, Landsburg D J, Carfi A, Wiley D C, Eisenberg R J, Cohen G H. (2002) J Virol. 76, 10894-904.
11. Connolly S A, Landsburg D J, Carfi A, Wiley D C, Cohen G H, Eisenberg R J. (2003). J Virol. 77, 8127-40.
12. Yoon, M. & Spear, P. G. (2004) Proc. Natl. Acad. Sci. USA 101, 17252-17257.
17. Zhou, G., Avitabile, E., Campadelli-Fiume, G. & Roizman, B. (2003) J. Virol 77, 3759-3767.
18. Zhou, G., Ye, G. J., Debinski, W. & Roizman, B. (2002) Proc. Natl. Acad. Sci. USA 99, 15124-15129.
19. Kamiyama H, Zhou G, Roizman B. (2006) Gene Ther. 13, 621-9.
20. Zhou G, Roizman B. (2006). Proc Natl Acad Sci USA. 103, 5508-13.
21. Chou, J., Poon A. P. W., Johnson, J., and Roizman. (1994). J. Virol, 68, 8.304-8.311.
23. Gianni T, Menotti L, Campadelli-Fiume G. (2005) J Virol. 79, 7042-9.
25. Martinez W M, Spear P G. (2002). J Virol. 76, 7255-62.
Zhou G, et al., Proc. Natl. Acad. Sci. USA, 99(23):15124-9 (2002)
Ploug M, et al., J. Biol. Chem., 266:1926-33 (1991)
Moller J V, et al., J. Biol. Chem., 268:18659-72 (1993)
Cantero D, et al. Br. J. Cancer, 75:388-95 (1997)
Reuning U, et al., Int. J. Oncol., 13:893-906 (1998)
Wei Y, et al., Biol. Chem., 269: 32380-32388 (1994)
Fowler B, et al., Thromb. Haemost., 80: 148-154 (1998)
Roldan A L, et al., EMBO J., 9: 467-474 (1990)
Barnathan E S, et al., J. Biol. Chem., 265:2865-2872 (1990)

1. A herpes simplex virus comprising a first polynucleotide encoding a gD polypeptide fragment comprising amino acids 219-369 of SEQ ID NO:26 and a second polynucleotide encoding a targeting peptide, wherein said targeting peptide specifically interacts with the gD polypeptide fragment.

2. The herpes simplex virus according to claim 1 wherein the targeting peptide is a urokinase plasminogen activator peptide that specifically interacts with urokinase plasminogen activator receptor.

3. The herpes simplex virus according to claim 1 wherein the second polynucleotide further encodes a second gD polypeptide fragment comprising at least 28 contiguous amino acids of SEQ ID NO:26 and having a C-terminus at position 60 of SEQ ID NO:26.

4. (canceled)

5. (canceled)

6. (canceled)

7. A herpes simplex virus comprising a polynucleotide encoding a gD polypeptide fragment comprising amino acids 219-369 of SEQ ID NO:26 and a targeting peptide, wherein said targeting peptide is a member of a binding pair that specifically interacts with the other member of said binding pair.

8. (canceled)

9. (canceled)

10. The herpes simplex virus according to claim 7 wherein said polynucleotide further encodes a second gD polypeptide fragment comprising at least 28 contiguous amino acids of SEQ ID NO:26 and having a C-terminus at position 60 of SEQ ID NO:26.

11. (canceled)

12. (canceled)

13. (canceled)

14. (canceled)

15. The herpes simplex virus according to claim 7 wherein the targeting peptide is selected from the group consisting of a urokinase plasminogen activator peptide fragment and an interleukin 13 peptide fragment, wherein said peptide fragment specifically interacts with its binding partner.

16. The herpes simplex virus according to claim 1 wherein the targeting peptide comprises an antibody variable domain, wherein said peptide specifically interacts with its binding partner.

17. A pharmaceutical composition comprising the herpes simplex virus according to claim 1 and a pharmaceutically acceptable excipient, carrier or diluent.

18. A kit comprising the herpes simplex virus according to claim 1 and a label providing instruction for administration of said virus.

19. A method of producing a herpes simplex virus according to claim 1 comprising:
(a) contacting a permissive host cell with the herpes simplex virus;
(b) incubating said host cell; and
(c) recovering said herpes simplex virus.

20. A method of treating a condition in an organism characterized by the presence of a deleterious cell in said organism comprising administering a therapeutically effective amount of a herpes simplex virus according to claim 1 to said organism.

21. The method according to claim 20 wherein said condition is cancer.

22. (canceled)

23. (canceled)

24. (canceled)

25. The herpes simplex virus according to claim 7 wherein the targeting peptide comprises an antibody variable domain, wherein said peptide specifically interacts with its binding partner.

26. A pharmaceutical composition comprising the herpes simplex virus according to claim 7 and a pharmaceutically acceptable excipient, carrier or diluent.

27. A kit comprising the herpes simplex virus according to claim 7 and a label providing instruction for administration of said virus.

28. A method of producing a herpes simplex virus according to claim 7 comprising:
(a) contacting a permissive host cell with the herpes simplex virus;
(b) incubating said host cell; and
(c) recovering said herpes simplex virus.

29. A method of treating a condition in an organism characterized by the presence of a deleterious cell in said organism comprising administering a therapeutically effective amount of a herpes simplex virus according to claim 7 to said organism.

30. The method according to claim 29 wherein said condition is cancer.
